(19) United States
(12) Patent Application Publication
(10) Pub. No.: US 2015/0010898 A1
(43) Pub. Date: Jan. 8, 2015

(54) METHODS FOR PREPARATION OF CULTURE MEDIA

(71) Applicant: Shire Human Genetic Therapies, Inc., Lexington, MA (US)

(72) Inventor: Robin Ng, Lexington, MA (US)

(73) Assignee: Shire Human Genetic Therapies, Inc., Lexington, MA (US)

(21) Appl. No.: 14/354,778

(22) PCT Filed: Oct. 26, 2012
(86) PCT No.: PCT/US12/62297

§ 371 Date: Apr. 28, 2014

Related U.S. Application Data
(60) Provisional application No. 61/553,132, filed on Oct. 28, 2011.

(51) Int. Cl. C12N 9/16

(52) U.S. Cl. CPC C12N 9/16; C12Y 301/06001
USPC 435/3; 435/196; 435/404

(57) ABSTRACT

Methods for preparation of culture media and production of recombinant proteins described herein.

### CROSS REFERENCE TO RELATED APPLICATIONS

This application claims priority to U.S. Application Ser. No. 61/553,132, filed on Oct. 28, 2011. The disclosure of the prior application is considered part of (and is incorporated by reference in) the disclosure of this application.

### BACKGROUND

Cell culture is a complex process in which cells are grown under controlled conditions. Cell culture media is one of the key components in developing a robust cell culture process, for example, for production of recombinant proteins such as lysosomal enzyme arylsulfatase A (ASA). Ausosomal recessive genetic defect in ASA is the cause of metachromatic leukodystrophy (MLD). Defects in ASA can result in a progressive breakdown of membranes of the myelin sheath (demyelination) and accumulation of galactosyl sulphatide (cerebroside sulfate) in the white matter of both central nervous system (CNS) and peripheral nervous system.

### SUMMARY

The disclosure provides, in part, methods of preparing culture media and methods of culturing cells. Described herein are also methods of producing recombinant proteins (e.g., arylsulfatase A (ASA)).

The methods described herein can be used to evaluate or monitor starting materials, intermediates, final products, and processes in the production of proteins (e.g., ASA), or to ensure or predict the efficacy of protein production. For example, the methods described herein can be used to reduce batch-to-batch variations in recombinant protein (e.g., ASA) production, e.g., caused at least in part by batch-to-batch variations in culture media preparation. Therefore, the methods disclosed herein are useful, e.g., from a process standpoint, e.g., to monitor or ensure batch-to-batch consistency or quality, to determine whether to accept or reject a batch of protein preparation, or to guide or control of a step in protein production, e.g., by evaluating a characteristic associated with protein production (e.g., associated with culture media or culture media preparation) to a reference, e.g., a preselected value. For example, the presence, distribution, or amount of one or more characteristics described herein can be correlated with, and therefore can be used to evaluate, monitor, or predict cell growth (e.g., cell viability, viable cell density, or metabolic rate) and/or the quality, quantity, efficiency, or consistency of protein production (e.g., yield, activity, glycosylation, integrity, stability, or folding). The methods described herein are also useful for quality control and/or bioequivalence studies of protein (e.g., ASA) preparations.

In one aspect, the disclosure features a method of preparing a culture medium. For example, the culture medium can have one or more of the characteristics described herein, e.g., a pH or osmolality, or a range of pH or osmolality described herein, or the culture medium is prepared by a method described herein, such that optimal cell growth (e.g., cell viability, viable cell density, or metabolic rate) is achieved and/or the quality, quantity, efficiency, or consistency of protein production (e.g., yield, activity, glycosylation, integrity, stability, or folding) is ensured.

In some embodiments, at least about 50%, e.g., at least about 60%, 70%, 80%, 90%, 95%, or 99%, e.g., between about 50% and 95% (e.g., between about 70% and about 90%) of the protein (e.g., ASA) produced by the cell or cell culture has a preselected characteristic or value associated with cell growth (e.g., cell viability, viable cell density, or metabolic rate) and/or protein production (e.g., yield, activity, glycosylation, integrity, stability, or folding), e.g., a characteristic or value described herein.

Exemplary characteristics or values for a protein (e.g., ASA) can include, but not limited to, a preselected level of specific activity (e.g., arylsulfatase activity), e.g., between about 1 and about 500 U/mg, e.g., between about 5 and 250 U/mg, between about 50 and about 140 U/mg, between about 50 and about 100 U/mg, or between about 100 and about 140 U/mg; a preselected value for disulfide linkage formation, e.g., one, two, three, or more of unpaired cysteine residues, single disulfide, nested disulfide, and cystine knots; a preselected value for formylglycine, e.g., at position 51 of ASA; a preselected value for average molecular weight; a preselected value for glycosylation, e.g., type of glycans, e.g., sialylation, phosphorylation; and a set of preselected values for molecular weight distribution, e.g., based on glycosylation.

In some embodiments, the pH of the culture medium is between about 7.0 and about 7.6, e.g., between about 7.2 and about 7.4, e.g., about 7.3.

In some embodiments, the osmolality of the culture medium is between about 275 Osm/L and about 395 Osm/L, e.g., about 305 Osm/L and about 365 Osm/L, e.g., about 335 Osm/L.

In some embodiments, the culture media preparation comprises one or more steps of dissolving media powder in water, adjusting media pH, and/or adjusting media osmolality.

In some embodiments, the mixing time for dissolving media powder in water (e.g., from media powder addition to first media pH or osmolality adjustment) is between about 0.5 hour and about 2 hours, e.g., about 1 hour and about 1.5 hours.

In some embodiments, the media pH is adjusted based on the following equation: Final pH=Initial pH+{(7.3−Initial pH)×(0.95±0.02)}, e.g., Final pH=Initial pH+{(7.3−Initial pH)×0.95}. In some embodiments, the initial pH is between about 7.0 and about 7.2, e.g., between about 7.02 and about 7.14, e.g., about 7.08. In some embodiments, the final pH is between about 7.2 and 7.4, e.g., about 7.3. In some embodiments, media pH is not adjusted based on the following equation: Final pH=Initial pH+[(7.3−Initial pH)×0.75].

In some embodiments, adjusting media pH is performed prior to adjusting media osmolality. In some embodiments, adjusting media pH is performed after adjusting media osmolality.

In some embodiments, the method further comprises mixing the culture medium after adjusting media pH, e.g., for about 10 minutes to about 1 hour, e.g., about 15 minutes to about 30 minutes. In some embodiments, the method further comprises mixing the culture medium after adjusting media osmolality, e.g., for about 10 minutes to about 1 hour, e.g., about 15 minutes to about 30 minutes.

In some embodiments, the method comprises improving the process capability of the culture media preparation.

In some embodiments, the process capability is determined by a process capability index (e.g., Cpk) and/or a process performance index (e.g., Ppk). In some embodiments, the process capability index or process performance index is for one or more characteristics of the culture media preparation, e.g., media pH, media osmolality, or media preparation time.

In some embodiments, the process capability index or process performance index is improved by about 50%, 100%, 150%, 200%, 250%, 300%, 350%, 400%, 450%, 500%, 750%, 1000%, or more, e.g., as compared to the process capability index or process performance index measured prior to using a method described herein, e.g., Six Sigma approach and/or Monte Carlo simulation. In some embodiments, the process capability index or process performance index is improved to about 1.00, 1.20, 1.40, 1.60, 1.80, 2.00, 2.20, 2.40, 2.60, 2.80, 3.00, or more, e.g., improved from about 0.65 to about 2.50. In some embodiments, the process capability (Cpk) for pH (e.g., final pH) is greater than about 1.00, e.g., improved from equal to or less than about 0.31. In some embodiments, the process capability (Cpk) for osmolality (e.g., final osmolality) is greater than about 1.80.

In some embodiments, the process capability is improved to increase the likelihood that the preparation results in a medium that meets one or more specifications (e.g., pH, osmolality); to reduce the amount of time used for media preparation; and/or to reduce the amount of time used to adjust one or more media parameters (e.g., pH, osmolality), e.g., by reducing or minimizing secondary adjustment for one or more media parameters. In some embodiments, the specifications comprise a pH between about 7.0 and about 7.6 (e.g., between about 7.2 and about 7.4, e.g., about 7.3) and/or osmolality between about 275 Osm/L and about 395 Osm/L (e.g., about 305 Osm/L and about 365 Osm/L, e.g., about 335 Osm/L).

In some embodiments, improving the process capability comprises defining one or more characteristics of the culture media preparation. In some embodiments, the characteristics comprise media pH, media osmolality, media preparation time, or a combination thereof.

In some embodiments, improving the process capability further comprises measuring one or more parameters associated with the culture media preparation, e.g., associated with media powder dissolution, pH adjustment, and/or osmolality adjustment.

In some embodiments, the parameter associated with media powder dissolution is selected from the group consisting of type of medium, mixing time (e.g., from media powder addition to first media pH or osmolality adjustment), type of agitator, type of media preparation protocol, component addition sequence, weight, and water quality.

In some embodiments, the parameter associated with pH adjustment is selected from the group consisting of: initial pH following the dissolution, mixing time from powder addition to first pH measurement, pH measurement device used to measure pH, concentration of sodium hydroxide (NaOH) used, amount of sodium hydroxide (NaOH) used; mixing time after the pH adjustment, equation used to predict the amount of sodium hydroxide needed, and type of medium.

In some embodiments, the parameter associated with osmolality adjustment is selected from the group consisting of: initial osmolality of the medium following the dissolution, type of medium, osmometer used to measure the medium osmolality, amount of sodium chloride used to adjust the osmolality, amount of sodium hydroxide used to adjust the pH, amount of hydrochloric acid used to adjust the pH, mixing time from powder addition to first osmolality measurement, and mixing time after the adjustment.

In some embodiments, measuring one or more parameters comprises performing a failure mode and effect analysis, e.g., a failure mode and effect analysis described herein. In some embodiments, measuring one or more parameters comprise performing a measurement system analysis, e.g., a measurement system analysis described herein.

In some embodiments, improving the process capability further comprises analyzing one or more parameters associated with the culture media preparation. In some embodiments, analyzing one or more parameters comprises performing a historical data analysis, a multivariate analysis, a Monte Carlo simulation, or a combination thereof.

In some embodiments, improving the process capability further comprises improving one or more parameters associated with the culture media preparation. In some embodiments, improving one or more parameters comprises: improving the pH measurement, e.g., by using only one pH measurement system, e.g., for both a first (e.g., initial) pH reading and a second (e.g., final) pH reading; improving the total mixing time, e.g., between dissolution and the first pH or osmolality measurement, e.g., to be between 0.5 hour and 3.0 hours, e.g., between 1 hour and 1.5 hour; improving the adjustment time, e.g., the mixing time after the pH or osmolality adjustment, e.g., between the pH measurement and the osmolality adjustment, e.g., to be between 5 and 60 minutes, e.g., between 15 and 30 minutes; improving the pH adjustment guideline equation to, e.g., amount of 10 N NaOH added equals to (7.3−initial pH)×0.90; or a combination thereof.

In some embodiments, improving the process capability further comprises controlling one or more parameters associated with the culture media preparation, e.g., to ensure the improvements are sustained. In some embodiments, controlling one or more parameters comprises: correcting for the pH adjustment equation in the medium preparation protocol, e.g., as described herein; controlling and/or recording the medium preparation mixing time, e.g., between dissolution and the first pH or osmolality measurement, e.g., to be between 0.5 and 3.0 hours, e.g., between 1 and 1.5 hours; controlling and/or recording the mixing time after the medium pH or osmolality adjustment, e.g., to be between 5 and 60 minutes, e.g., between 15 and 30 minutes; monitoring the medium osmolality after the pH adjustment; or a combination thereof. In some embodiments, controlling one or more parameters further comprises: monitoring the capability of the pH adjustment equation to adjust pH within specs; monitoring the variation of the initial pH after the improvement; monitoring the variation of pH adjustment equation after controlling the pH adjustment time; or a combination thereof.

In some embodiments, the medium is a pre-filtered medium. In some embodiments, the medium is for culturing a eukaryotic cell, e.g., an animal cell, e.g., a mammalian cell, e.g., a human cell. In some embodiments, the cell is a primary cell. In some embodiments, the cell is a secondary cell. In some embodiments, the cell is a fibroblast cell, e.g., an HT-1080 cell. In some embodiments, the medium is for culturing a cell strain. In some embodiments, the medium is for culturing a cell line. In some embodiments, the medium is a cell expansion medium. In some embodiments, the medium is a growth and production medium.

In some embodiments, the culture media preparation further comprises the step of adding serum (e.g., heat inactivated fetal bovine serum) to the medium. In some embodiments, the culture media preparation further comprises the step of adding one or more amino acid supplements (e.g., L-glutamine) to the medium. In some embodiments, the culture media preparation further comprises the step of adding one or more antibiotics (e.g., antibacterials or antifungals) to the medium. In some embodiments, the culture media preparation further comprises the step of adding one or more growth factors to the medium. In some embodiments, the culture media preparation further comprises the step of filtering or sterilizing the medium. In some embodiments, the culture media preparation further comprises the step of storing the medium, e.g., at room temperature or at about 4° C.

In another aspect, the disclosure features a method of producing a recombinant protein (e.g., arylsulfatase A (ASA)), the method comprising: preparing a culture medium by a method described herein; and culturing a cell under conditions that allow the expression of the recombinant protein (e.g., ASA).

The culture medium can have one or more of the characteristics described herein, e.g., a pH or osmolality, or a range of pH or osmolality described herein, or the culture medium is prepared by a method described herein, such that optimal cell growth (e.g., cell viability, viable cell density, or metabolic rate) is achieved and/or the quality, quantity, efficiency, or consistency of protein production (e.g., yield, activity, glycosylation, integrity, stability, or folding) is ensured.

In some embodiments, at least about 50%, e.g., at least about 60%, 70%, 80%, 90%, 95%, or 99%, e.g., between about 50% and 95% (e.g., between about 70% and about 90%) of the protein (e.g., ASA) produced by the cell or cell culture has a preselected characteristic or value associated with cell growth (e.g., cell viability, viable cell density, or metabolic rate) and/or protein production (e.g., yield, activity, glycosylation, integrity, stability, or folding), e.g., a characteristic or value described herein.

Exemplary characteristics or values for a protein (e.g., ASA) can include, but not limited to, a preselected level of specific activity (e.g., arylsulfatase activity), e.g., between about 1 and about 500 U/mg, e.g., between about 5 and about 250 U/mg, between about 50 and about 140 U/mg, between about 50 and about 100 U/mg, or between about 100 and about 140 U/mg; a preselected value for disulfide linkage formation, e.g., one, two, three, or more of unpaired cysteine residues, single disulfide, nested disulfide, and cystine knots; a preselected value for formylglycine, e.g., at position 51 of ASA; a preselected value for average molecular weight; a preselected value for glycosylation, e.g., type of glycans, e.g., sialylation, phosphorylation; and a set of preselected values for molecular weight distribution, e.g., based on glycosylation.

In some embodiments, the pH of the culture medium is between about 7.0 and about 7.6, e.g., between about 7.2 and about 7.4, e.g., about 7.3.

In some embodiments, the osmolality of the culture medium is between about 275 Osm/L and about 395 Osm/L, e.g., about 305 Osm/L and about 365 Osm/L, e.g., about 335 Osm/L.

In some embodiments, the culture media preparation comprises one or more steps of dissolving media powder in water, adjusting media pH, and/or adjusting media osmolality.

In some embodiments, the mixing time for dissolving media powder in water (e.g., from media powder addition to first media pH or osmolality adjustment) is between about 0.5 hour and about 2 hours, e.g., about 1 hour and about 1.5 hours.

In some embodiments, media pH is adjusted based on the following equation: Final pH=Initial pH+{(7.3−Initial pH)×(0.95±0.02)}, e.g., Final pH=Initial pH+{(7.3−Initial pH)×0.95}. In some embodiments, the initial pH is between about 7.0 and about 7.2, e.g., between about 7.02 and about 7.14, e.g., about 7.08. In some embodiments, the final pH is between about 7.2 and 7.4, e.g., about 7.3. In some embodiments, media pH is not adjusted based on the following equation: Final pH=Initial pH+[(7.3−Initial pH)×0.75].

In some embodiments, adjusting media pH is performed prior to adjusting media osmolality. In some embodiments, adjusting media pH is performed after adjusting media osmolality.

In some embodiments, the method further comprises mixing the culture medium after adjusting media pH, e.g., for about 10 minutes to about 1 hour, e.g., about 15 minutes to about 30 minutes. In some embodiments, the method further comprises mixing the culture medium after adjusting media osmolality, e.g., for about 10 minutes to about 1 hour, e.g., about 15 minutes to about 30 minutes.

In some embodiments, the method comprises improving the process capability of the culture media preparation. In some embodiments, the process capability is determined by a process capability index (e.g., Cpk) and/or a process performance index (e.g., Ppk). In some embodiments, the process capability index or process performance index is for one or more characteristics of the culture media preparation, e.g., media pH, media osmolality, or media preparation time.

In some embodiments, the process capability index or process performance index is improved by about 50%, 100%, 150%, 200%, 250%, 300%, 350%, 400%, 450%, 500%, 750%, 1000%, or more, e.g., as compared to the process capability index or process performance index measured prior to using a method described herein, e.g., Six Sigma approach and/or Monte Carlo simulation. In some embodiments, the process capability index or process performance index is improved to about 1.00, 1.20, 1.40, 1.60, 1.80, 2.00, 2.20, 2.40, 2.60, 2.80, 3.00, or more, e.g., improved from about 0.65 to about 2.50. In some embodiments, the process capability (Cpk) for pH (e.g., final pH) is greater than about 1.00, e.g., improved from equal to or less than about 0.31. In some embodiments, the process capability (Cpk) for osmolality (e.g., final osmolality) is greater than about 1.80.

In some embodiments, the process capability is improved to increase the likelihood that the preparation results in a medium that meets one or more specifications (e.g., pH, osmolality); to reduce the amount of time used for media preparation; and/or to reduce the amount of time used to adjust one or more media parameters (e.g., pH, osmolality), e.g., by reducing or minimizing secondary adjustment for one or more media parameters. In some embodiments, the specifications comprise a pH between about 7.0 and about 7.6 (e.g., between about 7.2 and about 7.4, e.g., about 7.3) and/or osmolality between about 275 Osm/L and about 395 Osm/L (e.g., about 305 Osm/L and about 365 Osm/L, e.g., about 335 Osm/L).

In some embodiments, improving the process capability comprises defining one or more characteristics of the culture media preparation. In some embodiments, the characteristics comprise media pH, media osmolality, media preparation time, or a combination thereof.

In some embodiments, improving the process capability further comprises measuring one or more parameters associated with the culture media preparation, e.g., associated with media powder dissolution, pH adjustment, and/or osmolality adjustment.

In some embodiments, the parameter associated with media powder dissolution is selected from the group consisting of type of medium, mixing time (e.g., from media powder addition to first media pH or osmolality adjustment), type of agitator, type of media preparation protocol, component addition sequence, weight, and water quality.

In some embodiments, the parameter associated with pH adjustment is selected from the group consisting of: initial pH following the dissolution, mixing time from powder addition to first pH measurement, pH measurement device used to measure pH, concentration of sodium hydroxide (NaOH) used, amount of sodium hydroxide (NaOH) used; mixing time after the pH adjustment, equation used to predict the amount of sodium hydroxide needed, and type of medium.

In some embodiments, the parameter associated with osmolality adjustment is selected from the group consisting of: initial osmolality of the medium following the dissolution, type of medium, osmometer used to measure the medium osmolality, amount of sodium chloride used to adjust the osmolality, amount of sodium hydroxide used to adjust the pH, amount of hydrochloric acid used to adjust the pH, mixing time from powder addition to first osmolality measurement, and mixing time after the adjustment.

In some embodiments, measuring one or more parameters comprises performing a failure mode and effect analysis, e.g., a failure mode and effect analysis described herein. In some embodiments, measuring one or more parameters comprise performing a measurement system analysis, e.g., a measurement system analysis described herein.

In some embodiments, improving the process capability further comprises analyzing one or more parameters associated with the culture media preparation. In some embodiments, analyzing one or more parameters comprises performing a historical data analysis, a multivariate analysis, a Monte Carlo simulation, or a combination thereof.

In some embodiments, improving the process capability further comprises improving one or more parameters associated with the culture media preparation. In some embodiments, improving one or more parameters comprises: improving the pH measurement, e.g., by using only one pH measurement system, e.g., for both a first (e.g., initial) pH reading and a second (e.g., final) pH reading; improving the total mixing time, e.g., between dissolution and the first pH or osmolality measurement, e.g., to be between 0.5 hour and 3.0 hours, e.g., between 1 hour and 1.5 hour; improving the adjustment time, e.g., the mixing time after the pH or osmolality adjustment, e.g., between the pH measurement and the osmolality adjustment, e.g., to be between 5 and 60 minutes, e.g., between 15 and 30 minutes; improving the pH adjustment guideline equation to, e.g., amount of 10 N NaOH added equals to (7.3−initial pH)×0.90; or a combination thereof.

In some embodiments, improving the process capability further comprises controlling one or more parameters associated with the culture media preparation, e.g., to ensure the improvements are sustained. In some embodiments, controlling one or more parameters comprises: correcting for the pH adjustment equation in the medium preparation protocol, e.g., as described herein; controlling and/or recording the medium preparation mixing time, e.g., between dissolution and the first pH or osmolality measurement, e.g., to be between 0.5 and 3.0 hours, e.g., between 1 and 1.5 hours; controlling and/or recording the mixing time after the medium pH or osmolality adjustment, e.g., to be between 5 and 60 minutes, e.g., between 15 and 30 minutes; monitoring the medium osmolality after the pH adjustment; or a combination thereof. In some embodiments, controlling one or more parameters further comprises: monitoring the capability of the pH adjustment equation to adjust pH within specs; monitoring the variation of the initial pH after the improvement; monitoring the variation of pH adjustment equation after controlling the pH adjustment time; or a combination thereof.

In some embodiments, the medium is a pre-filtered medium. In some embodiments, the medium is a cell expansion medium. In some embodiments, the medium is a growth and production medium.

In some embodiments, the culture media preparation further comprises the step of adding serum (e.g., heat inactivated fetal bovine serum) to the medium. In some embodiments, the culture media preparation further comprises the step of adding one or more amino acid supplements (e.g., L-glutamine) to the medium. In some embodiments, the culture media preparation further comprises the step of adding one or more antibiotics (e.g., antibacterials or antifungals) to the medium. In some embodiments, the culture media preparation further comprises the step of adding one or more growth factors to the medium. In some embodiments, the culture media preparation further comprises the step of filtering or sterilizing the medium. In some embodiments, the culture media preparation further comprises the step of storing the medium, e.g., at room temperature or at about 4° C.

In some embodiments, the cell is a eukaryotic cell, e.g., an animal cell, e.g., a mammalian cell, e.g., a human cell. In some embodiments, the cell is a primary cell or a secondary cell. In some embodiments, the cell is a fibroblast cell, e.g., an HT-1080 cell. In some embodiments, the cell is a cell strain. In some embodiments, the cell is a cell line.

In some embodiments, the cell is genetically modified, e.g., transfected with a mammalian expression vector construct, or transfected with a nucleic acid coding for a regulatory element controlling the expression of the coding sequence for the protein (e.g., ASA).

In some embodiments, the method further includes obtaining the recombinant protein (e.g., ASA) from the culture medium and/or from the cell.

In some embodiments, the method further comprises purifying the recombinant protein (e.g., ASA).

The methods described herein can also be used to select a batch of a protein (e.g., ASA) preparation, e.g., from a plurality of batches of a protein preparation (e.g., ASA), each of the plurality having a batch-to-batch variation in one or more characteristics associated with protein production, e.g., protein yield, activity, glycosylation, integrity, stability, and folding. In some embodiments, the method includes selecting a batch that was produced by culturing cells in a culture medium having one or more characteristics described herein, e.g., a pH between about 7.0 and about 7.6 (e.g., between about 7.2 and about 7.4, e.g., about 7.3), or osmolality between about 275 Osm/L and about 395 Osm/L (e.g., about 305 Osm/L and about 365 Osm/L, e.g., about 335 Osm/L), or a culture medium prepared by a method described herein, e.g., using a pH adjustment equation described herein, media preparation mixing time between about 1 hour and about 1.5 hours, mixing time after the media pH or osmolality adjustment between about 15 minutes and about 30 minutes, monitoring the media osmolality after the pH adjustment, or a combination thereof.

In yet another aspect, the disclosure features a method of culturing a cell, the method comprising: preparing a culture medium by a method described herein; and contacting a cell with the culture medium under conditions that allow culturing of the cell.

The culture medium can have one or more of the characteristics described herein, e.g., a pH or osmolality, or a range of pH or osmolality described herein, or the culture medium is prepared by a method described herein, such that optimal cell growth (e.g., cell viability, viable cell density, or metabolic rate) is achieved and/or the quality, quantity, efficiency, or consistency of protein production (e.g., yield, activity, glycosylation, integrity, stability, or folding) is ensured.

In some embodiments, at least about 50%, e.g., at least about 60%, 70%, 80%, 90%, 95%, or 99%, e.g., between about 50% and 95% (e.g., between about 70% and about 90%) of the protein (e.g., ASA) produced by the cell or cell culture has a preselected characteristic or value associated with cell growth (e.g., cell viability, viable cell density, or metabolic rate) and/or protein production (e.g., yield, activity, glycosylation, integrity, stability, or folding), e.g., a characteristic or value described herein.

Exemplary characteristics or values for a protein (e.g., ASA) can include, but not limited to, a preselected level of specific activity (e.g., arylsulfatase activity), e.g., between about 1 and about 500 U/mg, e.g., between about 5 and about 250 U/mg, between about 50 and about 140 U/mg, between about 50 and about 100 U/mg, or between about 100 and about 140 U/mg; a preselected value for disulfide linkage formation, e.g., one, two, three, or more of unpaired cysteine residues, single disulfide, nested disulfide, and cystine knots; a preselected value for formylglycine, e.g., at position 51 of ASA; a preselected value for average molecular weight; a preselected value for glycosylation, e.g., type of glycans, e.g., sialylation, phosphorylation; and a set of preselected values for molecular weight distribution, e.g., based on glycosylation.

In some embodiments, the pH of the culture medium is between about 7.0 and about 7.6, e.g., between about 7.2 and about 7.4, e.g., about 7.3.

In some embodiments, the osmolality of the culture medium is between about 275 Osm/L and about 395 Osm/L, e.g., about 305 Osm/L and about 365 Osm/L, e.g., about 335 Osm/L.

In some embodiments, the culture media preparation comprises one or more steps of dissolving media powder in water, adjusting media pH, and/or adjusting media osmolality.

In some embodiments, the mixing time for dissolving media powder in water (e.g., from media powder addition to first media pH or osmolality adjustment) is between about 0.5 hour and about 2 hours, e.g., about 1 hour and about 1.5 hours.

In some embodiments, the media pH is adjusted based on the following equation: Final pH=Initial pH+{(7.3−Initial pH)×(0.95±0.02)}, e.g., Final pH=Initial pH+{(7.3−Initial pH)×0.95}. In some embodiments, the initial pH is between about 7.0 and about 7.2, e.g., between about 7.02 and about 7.14, e.g., about 7.08. In some embodiments, the final pH is between about 7.2 and 7.4, e.g., about 7.3. In some embodiments, media pH is not adjusted based on the following equation: Final pH=Initial pH+[(7.3−Initial pH)×0.75].

In some embodiments, adjusting media pH is performed prior to adjusting media osmolality. In some embodiments, adjusting media pH is performed after adjusting media osmolality.

In some embodiments, the method further comprises mixing the culture medium after adjusting media pH, e.g., for about 10 minutes to about 1 hour, e.g., about 15 minutes to about 30 minutes. In some embodiments, the method further comprises mixing the culture medium after adjusting media osmolality, e.g., for about 10 minutes to about 1 hour, e.g., about 15 minutes to about 30 minutes.

In some embodiments, the method comprises improving the process capability of the culture media preparation. In some embodiments, the process capability is determined by a process capability index (e.g., Cpk) and/or a process performance index (e.g., Ppk). In some embodiments, the process capability index or process performance index is for one or more characteristics of the culture media preparation, e.g., media pH, media osmolality, or media preparation time.

In some embodiments, the process capability index or process performance index is improved by about 50%, 100%, 150%, 200%, 250%, 300%, 350%, 400%, 450%, 500%, 750%, 1000%, or more, e.g., from the process capability index or process performance index measured prior to using a method described herein, e.g., Six Sigma approach and/or Monte Carlo simulation. In some embodiments, the process capability index or process performance index is improved to about 1.00, 1.20, 1.40, 1.60, 1.80, 2.00, 2.20, 2.40, 2.60, 2.80, 3.00, or more, e.g., improved from about 0.65 to about 2.50. In some embodiments, the process capability (Cpk) for pH (e.g., final pH) is greater than about 1.00, e.g., improved from equal to or less than about 0.31. In some embodiments, the process capability (Cpk) for osmolality (e.g., final osmolality) is greater than about 1.80.

In some embodiments, the process capability is improved to increase the likelihood that the preparation results in a medium that meets one or more specifications (e.g., pH, osmolality); to reduce the amount of time used for media preparation; and/or to reduce the amount of time used to adjust one or more media parameters (e.g., pH, osmolality), e.g., by reducing or minimizing secondary adjustment for one or more media parameters. In some embodiments, the specifications comprise a pH between about 7.0 and about 7.6 (e.g., between about 7.2 and about 7.4, e.g., about 7.3) and/or osmolality between about 275 Osm/L and about 395 Osm/L (e.g., about 305 Osm/L and about 365 Osm/L, e.g., about 335 Osm/L).

In some embodiments, improving the process capability comprises defining one or more characteristics of the culture media preparation. In some embodiments, the characteristics comprise media pH, media osmolality, media preparation time, or a combination thereof.

In some embodiments, improving the process capability further comprises measuring one or more parameters associated with the culture media preparation, e.g., associated with media powder dissolution, pH adjustment, and/or osmolality adjustment.

In some embodiments, the parameter associated with media powder dissolution is selected from the group consisting of type of medium, mixing time (e.g., from media powder addition to first media pH or osmolality adjustment), type of agitator, type of media preparation protocol, component addition sequence, weight, and water quality.

In some embodiments, the parameter associated with pH adjustment is selected from the group consisting of: initial pH following the dissolution, mixing time from powder addition to first pH measurement, pH measurement device used to measure pH, concentration of sodium hydroxide (NaOH) used, amount of sodium hydroxide (NaOH) used; mixing time after the pH adjustment, equation used to predict the amount of sodium hydroxide needed, and type of medium.

In some embodiments, the parameter associated with osmolality adjustment is selected from the group consisting of: initial osmolality of the medium following the dissolution, type of medium, osmometer used to measure the medium osmolality, amount of sodium chloride used to adjust the osmolality, amount of sodium hydroxide used to adjust the pH, amount of hydrochloric acid used to adjust the pH, mixing time from powder addition to first osmolality measurement, and mixing time after the adjustment.

In some embodiments, measuring one or more parameters comprises performing a failure mode and effect analysis, e.g., a failure mode and effect analysis described herein. In some embodiments, measuring one or more parameters comprise performing a measurement system analysis, e.g., a measurement system analysis described herein.

In some embodiments, improving the process capability further comprises analyzing one or more parameters associated with the culture media preparation. In some embodiments, analyzing one or more parameters comprises performing a historical data analysis, a multivariate analysis, a Monte Carlo simulation, or a combination thereof.

In some embodiments, improving the process capability further comprises improving one or more parameters associated with the culture media preparation. In some embodiments, improving one or more parameters comprises: improving the pH measurement, e.g., by using only one pH measurement system, e.g., for both a first (e.g., initial) pH reading and a second (e.g., final) pH reading; improving the total mixing time, e.g., between dissolution and the first pH or osmolality measurement, e.g., to be between 0.5 hour and 3.0 hours, e.g., between 1 hour and 1.5 hour; improving the adjustment time, e.g., the mixing time after the pH or osmolality adjustment, e.g., between the pH measurement and the osmolality adjustment, e.g., to be between 5 and 60 minutes, e.g., between 15 and 30 minutes; improving the pH adjustment guideline equation to, e.g., amount of 10 N NaOH added equals to (7.3−initial pH)×0.90; or a combination thereof.

In some embodiments, improving the process capability further comprises controlling one or more parameters associated with the culture media preparation, e.g., to ensure the improvements are sustained. In some embodiments, controlling one or more parameters comprises: correcting for the pH adjustment equation in the medium preparation protocol, e.g., as described herein; controlling and/or recording the medium preparation mixing time, e.g., between dissolution and the first pH or osmolality measurement, e.g., to be between 0.5 and 3.0 hours, e.g., between 1 and 1.5 hours; controlling and/or recording the mixing time after the medium pH or osmolality adjustment, e.g., to be between 5 and 60 minutes, e.g., between 15 and 30 minutes; monitoring the medium osmolality after the pH adjustment; or a combination thereof. In some embodiments, controlling one or more parameters further comprises: monitoring the capability of the pH adjustment equation to adjust pH within specs; monitoring the variation of the initial pH after the improvement; monitoring the variation of pH adjustment equation after controlling the pH adjustment time; or a combination thereof.

In some embodiments, the medium is a pre-filtered medium. In some embodiments, the medium is a cell expansion medium. In some embodiments, the medium is a growth and production medium.

In some embodiments, the culture media preparation further comprises the step of adding serum (e.g., heat inactivated fetal bovine serum) to the medium. In some embodiments, the culture media preparation further comprises the step of adding one or more amino acid supplements (e.g., L-glutamine) to the medium. In some embodiments, the culture media preparation further comprises the step of adding one or more antibiotics (e.g., antibacterials or antifungals) to the medium. In some embodiments, the culture media preparation further comprises the step of adding one or more growth factors to the medium. In some embodiments, the culture media preparation further comprises the step of filtering or sterilizing the medium. In some embodiments, the culture media preparation further comprises the step of storing the medium, e.g., at room temperature or at about 4° C.

In some embodiments, the cell is a eukaryotic cell, e.g., an animal cell, e.g., a mammalian cell, e.g., a human cell. In some embodiments, the cell is a primary cell or a secondary cell. In some embodiments, the cell is a fibroblast cell, e.g., an HT-1080 cell. In some embodiments, the cell is a cell strain. In some embodiments, the cell is a cell line.

In some embodiments, the cell is genetically modified, e.g., transfected with a mammalian expression vector construct, or transfected with a nucleic acid coding for a regulatory element controlling the expression of the coding sequence for the protein (e.g., ASA).

In one aspect, the disclosure features a culture medium that has one or more of the characteristics described herein or is prepared by a method described herein. In another aspect, the disclosure features a cell cultured by a culture medium described herein. In yet another aspect, the disclosure features a protein (e.g., ASA) produced by a method described herein.

### BRIEF DESCRIPTION OF THE DRAWINGS

FIG. 1 depicts an initial run chart for pH.

FIG. 2 depicts an initial process capability for pH.

FIG. 3 depicts an initial run chart for osmolality.

FIG. 4 depicts an initial process capability for osmolality.

FIG. 5 depicts a high level process map.

FIG. 6 depicts a detailed process map.

FIG. 7 depicts a cause and effect matrix.

FIG. 8 depicts a failure mode and effect analysis.

FIG. 9 depicts a gage run chart of pH in the measurement system analysis.

FIG. 10 depicts the results for the measurement system analysis.

FIG. 11 is representative box-plots from the low pH, mid pH, and high pH samples in the measurement system analysis.

FIG. 12 depicts the correlation between sample pH and the pH difference in the measurement system analysis.

FIG. 13 depicts the correlation between sample pH and % Diff in the measurement system analysis.

FIG. 14 depicts the effects of preparation time on initial pH in the historical data analysis and multivariate analysis.

FIG. 15 depicts the effects of preparation time, adjustment time, and base added on (Final pH−Initial pH) in the historical data analysis and multivariate analysis.

FIG. 16 depicts the results for regression, residual analysis, and forced zero regression in the historical data analysis.

FIG. 17 depicts the results for Monte Carlo simulation.

FIG. 18 is an I-MR chart of initial pH by improvement.

FIG. 19 is an I-MR chart of preparation time by improvement.

FIG. 20 is an I-MR chart of adjustment time by improvement.

FIG. 21 is an I-MR chart of final pH by improvement.

FIG. 22 is capability histograms of final pH by improvement.

FIG. 23 depicts process capability of final pH after filtration.

### DETAILED DESCRIPTION

Described herein are methods of preparing culture media, methods of culturing cells, and methods of producing recombinant proteins (e.g., arylsulfatase A (ASA)).

### Cell Culture

Cell culture is the complex process by which cells are grown under controlled conditions. As used herein, the term “cell culture” refers to the culturing of cells or tissues derived from multi-cellular eukaryotes (e.g., animal cells (e.g., mammalian cells), plant cells) or microorganisms (e.g., bacteria, fungi, viruses).

As used herein, “primary cell” refers to a cell that is cultured directly from a subject; and “secondary cell” refers to a cell arising from multiplication of a primary cell.

As used herein, “cell strain” refers to cells adapted to culture, but with finite division potential; and “cell line” refers to cells that have acquired the ability to proliferate indefinitely, for example, through random mutation or deliberate modification, such as artificial expression of the telomerase gene.

Cells can be grown and maintained at an appropriate temperature and gas mixture (for example, 37° C., 5% CO₂) in a cell incubator. Culture conditions can vary for each cell type and variation of conditions (e.g., plating density) for a particular cell type can result in different phenotypes being expressed.

The methods described herein can be used for culturing various types of cells (e.g., animal cells). Exemplary types of animal cells include, but not limited to, fibroblasts, epithelial cells, neuronal cells, endothelial cells, stem cells, blood cells, lymphocytes, and muscle cells.

Cells can be grown either in suspension or adherent cultures. Some cells, such as cells that exist in the bloodstream, naturally live in suspension, without being attached to a surface. There are also cell lines that have been modified to be able to survive in suspension cultures so they can be grown to a higher density than adherent conditions would allow. Adherent cells require a surface, such as tissue culture plastic or microcarrier, which may be coated with extracellular matrix components to increase adhesion properties and provide other signals needed for growth and differentiation. Another type of adherent culture is organotypic culture, which involves growing cells in a three-dimensional (3-D) environment as opposed to two-dimensional culture dishes.

Common manipulations that can be carried out on culture cells include, e.g., media changes, passaging cells, and transfecting or infecting cells. These are generally performed using tissue culture methods that rely on sterile technique. Antibacterials (e.g. penicillin and streptomycin) and antifungals (e.g., amphotericin B) can also be added to the growth media.

As cells undergo metabolic processes, acid is produced and the pH decreases. Old media can be removed from adherent cells or spin-down suspension cells by aspiration and replaced with fresh media.

Passaging (also known as subculture or splitting cells) involves transferring a small number of cells into a new vessel. Cells can be cultured for a longer time if they are split regularly, as it avoids the senescence associated with prolonged high cell density. Suspension cultures can be passaged with a small amount of culture containing a few cells diluted in a larger volume of fresh media. For adherent cultures, cells first need to be detached. This can be done, e.g., by using a mixture of trypsin-EDTA. A small number of detached cells can then be used to seed a new culture.

Another common method for manipulating cells involves the introduction of foreign DNA by transfection. This can be performed to cause cells to express a protein of interest. The transfection of RNAi constructs can be used for suppressing the expression of a particular gene/protein. DNA can also be inserted into cells using viruses, in methods referred to as transduction, infection or transformation.

### Culture Medium

A culture medium or growth medium is a liquid or gel designed to support the growth of cells (e.g., animal cells, e.g., mammalian cells) or microorganisms. There are different types of media for growing different types of cells. There are two major types of growth media: those used for growing specific cell types derived from plants or animals, and those used for growing microorganisms, such as bacteria or yeast.

Recipes for culture media (e.g., expansion media or growth and production media) can vary, for example, in pH, glucose concentration, growth factors, and the presence of other nutrients. The growth factors used to supplement media are often derived from animal blood, such as calf serum. A pH indicator can be added to the medium to measure nutrient depletion as acid is produced when cells undergo metabolic process.

Exemplary media that can be used for mammalian cell culture include, but not limited to, BGJb (Fitton-Jackson Modification), BME, Brinster's BMOC-3, CMRL, CO2-Independent Medium, D-MEM Media, D-MEM/F-12 Media, F-10 Nutrient Mixture, F-12 Nutrient Mixture, Glasgow (G-MEM), Improved MEM, Iscove's (IMDM), Leibovitz's L-15, McCoy's 5A, MCDB 131, Media 199, Minimum Essential Media (MEM), Modified Eagle Medium (MEM), Opti-MEM® I, RPMI Medium 1640, Waymouth's MB 752/1, and Williams' Media E.

The culture media described herein can contain high concentration of glucose (e.g., about 4,500 mg/L), low concentration of glucose (e.g., about 1,000 mg/L), or no glucose.

The culture media described herein can contain one or more amino acids. Exemplary amino acids include, but not limited to, glycine, L-alanine, L-arginine, L-asparagine, L-aspartic acid, L-cystine, L-glutamic acid, L-histidine, L-isoleucine, L-leucine, L-lysine, L-methionine, L-phenylalanine, L-proline, L-serine, L-threonine, L-tryptophan, L-tyrosine, and L-valine. In some embodiments, the culture medium contains L-glutamine. In other embodiments, the culture medium does not contain L-glutamine.

The culture media described herein can contain one or more vitamins. Exemplary vitamins include, but not limited to, ascorbic acid phosphate, choline chloride, D-calcium pantothenate, folic acid, niacinamide, pyridoxine hydrochloride, riboflavin, thiamine hydrochloride, and i-inositol.

The culture media described herein can contain one or more inorganic salts. Exemplary inorganic salts include, but not limited to, calcium chloride (CaCl₂), ferric nitrate (Fe(NO₃)₃-9H₂O), magnesium sulfate (MgSO₄), potassium chloride (KCl), sodium bicarbonate (NaHCO₃), sodium chloride (NaCl), and sodium phosphate dibasic (Na₂HPO₄—H₂O).

The culture media described herein can contain one or more antibiotics or antifungals. Exemplary antibiotics and antifungals include, but not limited to, actinomycin D, amphotericin B, ampicillin, carbenicillin, cefotaxime, fosmidomycin, gentamicin, kanamycin, neomycin, penicillin, streptomycin, and polymyxin B.

The culture media described herein can contain one or more protein supplements. Exemplary protein supplements include, but not limited to, growth factors (activin, EGF, FGF), cytokines (e.g., G-CSF, GM-CSF, INF-α, INF-β), chemokines (e.g., CXCL-1, IL-8), human transferrin, and insulin.

In some embodiments, the culture medium contains serum. In other embodiments, the culture medium is serum-free.

### Six Sigma Approach

Six Sigma is a business management strategy as well as problem solving methodology to improve the quality of process outputs by identifying and minimizing variability in manufacturing and business processes. A typical Six Sigma approach can have the following five phases (“DMAIC”):

Define the problem, such as the project goals, specifically.

Measure key aspects of the current process and collect relevant data.

Analyze the data to investigate and verify cause-and-effect relationships. Determine what the relationships are, and attempt to ensure that all factors have been considered. Seek out root cause of the defect under investigation.

Improve or optimize the current process based upon data analysis using techniques such as design of experiments, mistake proofing, and standard work to create a new, future state process. Set up pilot runs to establish process capability.

Control the future state process to ensure that any deviations from target are corrected before they result in defects. Implement control systems such as statistical process control, production boards, visual workplaces, and continuously monitor the process.

Within the individual phases of a DMAIC project, Six Sigma can use one or more established quality-management tools that are also used outside of Six Sigma. Exemplary tools that can be used in the Six Sigma approach include, but not limited to, 5 Whys, accelerated life testing, analysis of variance, ANOVA Gauge R&R, axiomatic design, Business Process Mapping, cause & effects diagram (also known as fishbone or Ishikawa diagram), check sheet, Chi-squared test of independence and fits, control chart, correlation, cost-benefit analysis, CTQ tree, design of experiments, failure mode and effects analysis (FMEA), general linear model, histograms, Pareto analysis, Pareto chart, Pick chart, process capability, Quality Function Deployment (QFD), quantitative marketing research through use of Enterprise Feedback Management (EFM) systems, regression analysis, root cause analysis, run charts, scatter diagram, SIPOC analysis (Suppliers, Inputs, Process, Outputs, Customers), stratification, Taguchi methods, Taguchi Loss Function, and TRIZ.

### Process Capability

The Process Capability is a measurable property of a process to the specification, expressed as a process capability index (e.g., Cpk or Cpm) or as a process performance index (e.g., Ppk or Ppm). The output of this measurement can be illustrated by a histogram and calculations that predict how many parts will be produced out of specification (OOS). Parts of process capability can include, e.g., measurement of the variability of the output of a process, and comparison of the variability with a proposed specification or product tolerance.

The input of a process can have at least one or more measurable characteristics that are used to specify outputs. With a normal distribution of the output data, the process can be described by the process mean and the standard deviation. A control chart analysis can be used to determine whether the process is “in statistical control,” which indicates whether the process capability involves special cause variation. Generally, a batch of data needs to be obtained from the measured output of the process.

The output of a process is expected to meet customer requirements, specifications, or product tolerances. A process capability study can be conducted to determine the extent to which the process can meet these expectations. The ability of a process to meet specifications can be expressed as a single number using a process capability index or it can be assessed using control charts. Statistical process control defines techniques to properly differentiate between stable processes, processes that are drifting (experiencing a long-term change in the mean of the output), and processes that are growing more variable. Process capability indices are only meaningful for processes that are stable (in a state of statistical control).

### Process Capability Index

In process improvement efforts, the process capability index or process capability ratio is a statistical measure of process capability and shows the ability of a process to produce output within specification limits. Process capability indices can be used to measure how much “natural variation” a process experiences relative to its specification limits and allows different processes to be compared with respect to how well an organization controls them.

For example, Cₚ estimates what the process is capable of producing if the process mean were to be centered between the specification limits. C_{p,lower} estimates process capability for specifications that consist of a lower limit only (for example, strength). C_{p,upper} estimates process capability for specifications that consist of an upper limit only (for example, concentration). Cₚₖ estimates what the process is capable of producing, considering that the process mean may not be centered between the specification limits. Cₚₘ estimates process capability around a target. Cₚₖₘ estimates process capability around a target and accounts for an off-center process mean.

In particular, Cₚₖ can be defined as:

$${\hat{C}}_{p k} = min [ \frac{USL - \hat{μ}}{3 \hat{σ}} , \frac{\hat{μ} - LSL}{3 \hat{σ}} ]$$

wherein USL and LSL are the upper and lower specifications of the process, \hat{μ} is the estimated mean of the process, and \hat{σ} is the estimated variability of the process. σ̅ is estimated using the sample standard deviation.

### Process Performance Index

In process improvement efforts, the process performance index is an estimate of the process capability of a process during its initial set-up, before it has been brought into a state of statistical control. The process performance index can be defined as:

$${\hat{P}}_{p k} = min [ \frac{USL - \hat{μ}}{3 \times \hat{σ}} , \frac{\hat{μ} - LSL}{3 \times \hat{σ}} ]$$

wherein USL and LSL are the upper and lower specifications of the process, \hat{μ} is the estimated mean of the process, and \hat{σ} is the estimated variability of the process. \hat{σ} is estimated using the sample standard deviation. Larger values of Pₚₖ may be interpreted to indicate that a process is more capable of producing output within the specification limits.

### Failure Modes and Effects Analysis

A failure modes and effects analysis (FMEA) is a procedure in product development and operations management for analysis of potential failure modes within a system for classification by the severity and likelihood of the failures. A successful FMEA activity can be used to identify potential failure modes based on past experience with similar products or processes, enabling the team to design those failures out of the system with the minimum of effort and resource expenditure, thereby reducing development time and costs. Failure modes are any errors or defects in a process, design, or item, especially those that affect the customer, and can be potential or actual. Effects analysis refers to studying the consequences of those failures.

### Measurement System Analysis

A Measurement System Analysis (MSA) is a specially designed experiment that seeks to identify the components of variation in the measurement. A Measurement Systems Analysis evaluates the test method, measuring instruments, and the entire process of obtaining measurements to ensure the integrity of data used for analysis (e.g., quality analysis) and to understand the implications of measurement error for decisions made about a product or process. MSA is an element of Six Sigma methodology and of other quality management systems. Typically, MSA analyzes the collection of equipment, operations, procedures, software and personnel that affects the assignment of a number to a measurement characteristic. A Measurement Systems Analysis can consider the following: selecting the correct measurement and approach; assessing the measuring device; assessing procedures and operators; assessing any measurement interactions, calculating the measurement uncertainty of individual measurement devices and/or measurement systems. Common tools and techniques of Measurement Systems Analysis include, e.g., calibration studies, fixed effect ANOVA, components of variance, Attribute Gage Study, Gage R&R, ANOVA Gage R&R, Destructive Testing Analysis and others. The tool selected can be determined by characteristics of the measurement system itself.

### Multivariate Analysis

Multivariate analysis (MVA) is based on the statistical principle of multivariate statistics, which involves observation and analysis of more than one statistical variable at a time. For example, this technique can be used to perform trade studies across multiple dimensions while taking into account the effects of all variables on the responses of interest. Uses for multivariate analysis include, but not limited to, design for capability (capability-based design), inverse design (in which any variable can be treated as an independent variable), Analysis of Alternatives (AoA), analysis of concepts with respect to changing scenarios, and identification of critical design drivers and correlations across hierarchical levels. When physics-based analysis is desired to calculate the effects of variables for a hierarchical “system-of-systems,” surrogate models, which are highly accurate approximations of the physics-based code, can be used to enable large-scale MVA studies, for example, to enable a Monte Carlo simulation, which often takes the form of response surface equations.

### Monte Carlo Simulation

Monte Carlo simulation is a mathematic simulation based on the distribution of values for each input variable rather than a single fixed value. It calculates a probable range of output scenarios based on multiple iterative calculations using random values from within the defined probability ranges for each input variable.

### Production of Recombinant Proteins

Methods described herein can be used for producing recombinant proteins (e.g., arylsulfatase A).

### Arylsulfatase A

Arylsulfatase A (or cerebroside-sulfatase) is an enzyme that breaks down cerebroside 3-sulfate (or sulfatide) into cerebroside and sulfate. Specifically, galactosyl sulfatide is normally metabolized by the hydrolysis of 3-O-sulphate linkage to form galactocerebroside through the combined action of the lysosomal enzyme arylsulfatase A (EC 3.1.6.8) (Austin et al. Biochem J. 1964, 93, 15C-17C) and a sphingolipid activator protein called saposin B (Sap-B). A deficiency of arylsulfatase A occurs in all tissues from patients with the late infantile, juvenile, and adult forms of metachromatic leukodystrophy (MLD). As used herein, the arylsulfatase A protein will be termed “ASA”.

ASA is an acidic glucoprotein with a low isoelectric point. Above pH 6.5, the enzyme exists as a monomer with a molecular weight of approximately 100 kDa. ASA undergoes a pH-dependent polymerisation forming a dimer at pH 4.5. In human urine, the enzyme consists of two nonidentical subunits of 63 and 54 kDa (Laidler P M et al. Biochim Biophys Acta. 1985, 827, 73-83). ASA purified from human liver, placenta, and fibroblasts also consist of two subunits of slightly different sizes varying between 55 and 64 kDa (Draper R K et al. Arch Biochemica Biophys. 1976, 177, 525-538, Waheed A et al. Hoppe Seylers Z Physiol Chem. 1982, 363, 425-430, Fujii T et al. Biochim Biophys Acta. 1992, 15 1122, 93-98). As in the case of other lysosomal enzymes, ASA is synthesized on membrane-bound ribosomes as a glycosylated precursor. It then passes through the endoplasmic reticulum and Golgi, where its N-linked oligosaccharides are processed with the formation of phosphorylated and sulfated oligosaccharide of the complex type (Waheed A et al. Biochim Biophys Acta. 1985, 847, 53-61, Braulke T et al. Biochem Biophys Res Commun. 1987, 143, 178-185). In normal cultured fibroblasts, a precursor polypeptide of 62 kDa is produced, which translocates via mannose-6-phosphate receptor binding (Braulke T et al. J Biol. Chem. 1990, 265, 6650-6655) to an acidic prelysosomal endosome (Kelly B M et al. Eur J. Cell Biol. 1989, 48, 71-78).

The methods described herein can be used to purify ASA from any source, e.g., from tissues, or cultured cells (e.g., human cells (e.g., fibroblasts) that recombinantly produce ASA).

The length (18 amino acids) of the human ASA signal peptide is based on the consensus sequence and a specific processing site for a signal sequence. Hence, from the deduced human ASA cDNA (EMBL GenBank accession numbers J04593 and X521151) the cleavage of the signal peptide occurs in all cells after residue number 18 (Ala), resulting in the mature form of the human ASA. As used herein, recombinant arylsulfatase A will be abbreviated “rASA”. The mature form of arylsulfatase A including the mature form of human ASA will be termed “mASA” and the mature recombinant human ASA will be termed “mrhASA”.

Multiple forms of ASA have been demonstrated on electrophoresis and isoelectric focusing of enzyme preparations from human urine (Luijten JAFM et al. J Mol. Med. 1978, 3, 213), leukocytes (Dubois et al. Biomedicine. 1975, 23, 116-119, Manowitz P et al. Biochem Med Metab Biol. 1988, 39, 117-120), platelets (Poretz et al. Biochem J. 1992, 287, 979-983), cultured fibroblasts (Waheed A et al. Hoppe Seylers Z Physiol Chem. 1982, 363, 425-430, Stevens R L et al. Biochim Biophys Acta. 1976, 445, 661-671, Farrell D F et al. Neurology. 1979, 29, 16-20) and liver (Stevens R L et al. Biochim Biophys Acta. 1976, 445, 661-671, Farrell D F et al. Neurology. 1979, 29, 16-20, Sarafian T A et al. Biochem Med. 1985, 33, 372-380). Treatment with endoglycosidase H, sialidase, and alkaline phosphatase reduces the molecular size and complexity of the electrophoretic pattern, which suggests that much of the charge heterogeneity of ASA is due to variations in the carbohydrate content of the enzyme.

The active site of ASA contains an essential histidine residue (Lee G D and Van Etten R L, Arch Biochem Biophys. 1975, 171, 424-434) and two or more arginine residues (James G T, Arch Biochem Biophys. 1979, 97, 57-62). Many anions are inhibitors of the enzyme at concentrations in the millimolar range or lower.

A protein modification has been identified in two eukaryotic sulfatases (ASA and arylsulfatase B (ASB)) and for one from the green alga Volvox carteri (Schmidt B et al. Cell. 1995, 82, 271-278, Selmer T et al. Eur J. Biochem. 1996, 238, 341-345). This modification leads to the conversion of a cysteine residue, which is conserved among the known sulfatases, into a 2-amino-3-oxopropionic acid residue (Schmidt B et al. Cell. 1995, 82, 271-278). The novel amino acid derivative is also recognized as C-formylglycin (FGly). In ASA and ASB derived from MSD cells, the Cys-69 residue is retained. Consequently, it is proposed that the conversion of the Cys-69 to FGly-69 is required for generating catalytically active ASA and ASB, and that deficiency of this protein modification is the cause of MSD. Cys-69 is referred to the precursor ASA which has an 18 residue signal peptide. In the mASA the mentioned cysteine residue is Cys-51.

Further investigations have shown that a linear sequence of 16 residues surrounding the Cys-51 in the mASA is sufficient to direct the conversion and that the protein modification occurs after or at a late stage of co-translational protein translocation into the endoplasmic reticulum when the polypeptide is not yet folded to its native structure (Dierks T et al. Proc Natl Acad. Sci. 1997, 94, 11963-1196, Wittke, D. et al. (2004), Acta Neuropathol. (Berl.), 108, 261-271).

The human ASA gene structure has been described. As used herein, this gene will be termed “ARSA”. The ARSA gene is located near the end of the long arm of chromosome 22 (22ql3.31-qter), it spans 3.2 kb (Kreysing et al. Eur J. Biochem. 1990, 191, 627-631) and consists of eight exons specifying the 507 amino acid enzyme unit (Stein et al. J Biol. Chem. 1989, 264, 1252-1259). Messenger RNAs of 2.1, 3.7, and 4.8 kb have been detected in fibroblast cells, with the 2.1-kb message apparently responsible for the bulk of the active ASA generated by the cell (Kreysing et al. Eur J. Biochem. 1990, 191, 627-631). The ARSA sequence has been deposited at the EMBL GenBank with the accession number X521150. Differences between the published cDNA and the coding part of the ARSA were described by Kreysing et al. (Eur J. Biochem. 1990, 191, 627-631). The cDNA sequence originally described by Stein et al. (J Biol. Chem. 1989, 264, 1252-1259) and the cDNA sequence described by Kreysing et al. (Eur J Biochem. 1990, 191, 627-631) have been deposited at the EMBL GenBank with the following accession numbers J04593 and X521151, respectively.

Several polymorphisms and more than 40 disease-related mutations have been identified in the ARSA gene (Gieselmann et al. Hum Mutat. 1994, 4, 233-242, Barth et al. Hum Mutat. 1995, 6, 170-176, Draghia et al. Hum Mutat. 1997, 9, 234-242). The disease-related mutations in the ARSA gene can be categorised in two broad groups that correlate fairly well with the clinical phenotype of MLD. One group (I) produces no active enzyme, no immunoreactive protein, and expresses no ASA activity when introduced into cultured animal cell lines. The other group (A) generates small amounts of cross-reactive material and low levels of functional enzyme in cultured cells. Individuals homozygous for a group (I) mutation, or having two different mutations from this group, express late infantile MLD. Most individuals with one group (I)-type and one group (A)-type mutation develop the juvenile-onset form, whereas those with two group (A)-type mutations generally manifest adult MLD. Some of the mutations have been found relatively frequently, whereas others have been detected only in single families. It is possible to trace specific mutations through members of many families, however general carrier screening is not yet feasible.

In addition to the disease-related mutations described above, several polymorphisms have been identified in the ARSA gene. Extremely low ASA activity has been found in some clinically normal parents of MLD patients and also in the general population. This so-called pseudodeficiency ASA has been associated with a common polymorphism of the ARSA gene (Gieselmann et al. Dev Neurosci. 1991, 13, 222-227).

### EXAMPLES

### Example 1

### A Six Sigma Approach on Improving the Process Capability of Culture Media

Culture media preparation is one the initial steps for biologics manufacturing. Although specifications can be established to monitor the quality of the media prepared, secondary adjustment to the media may require more time and resource. The goal of this example is to improve the media preparation procedure so that the pre-filtered final parameters will meet the specifications. Specifically, the goals are to ensure that the media preparation step will result in media that meet the specifications; to reduce preparation time especially the amount of time to adjust media parameter; and to minimize secondary adjustment during media preparation.

In this example, historical data from the media preparation were trended and analyzed. A Six Sigma approach in combination with Monte Carlo simulation was applied to develop a more robust protocol. In addition to the establishment of the culture media specifications, key factors were identified for process monitoring.

### Specifications

Specifications for pre-filtered media after the preparation include: pH: 7.3+/−0.1 and osmolality: 335+/−30 Osm/L.

### Baseline Data

Initial: Improve the process capability (Cpk) for the final pH from 0.31 to >1.00, e.g., by shifting the average closer to the target value, and by striving to reduce variation during the media preparation procedure.

Since the final Osmolality Cpk is >1.80, the osmolality performance and stability will be monitored over time.

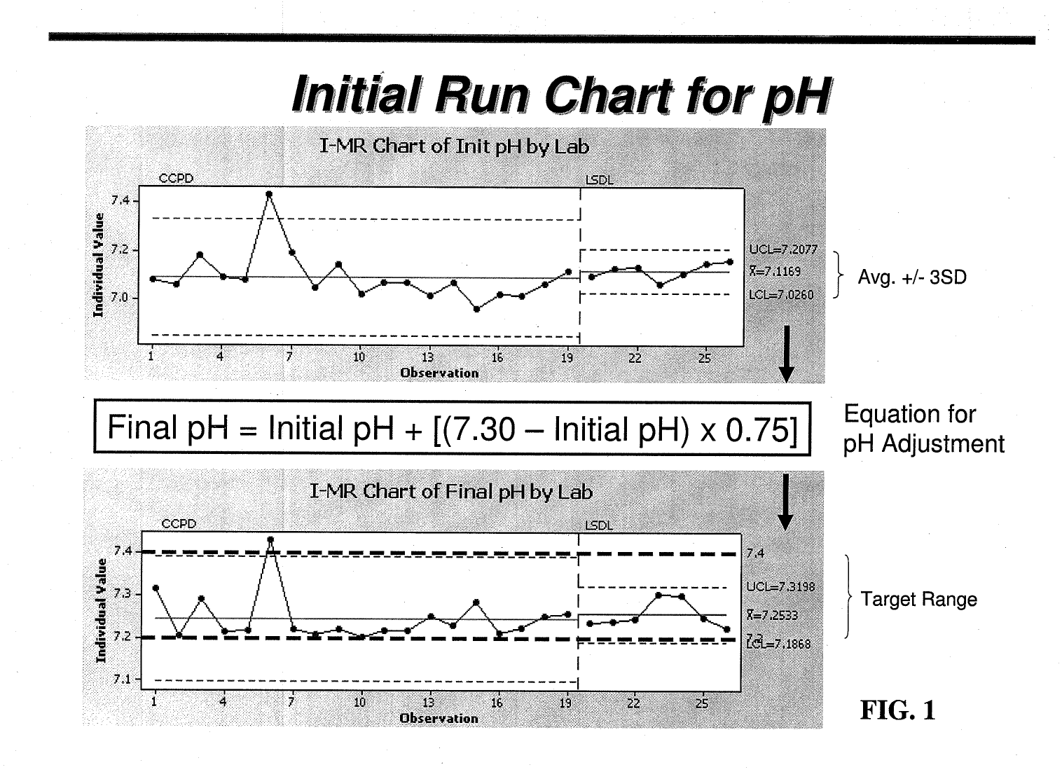

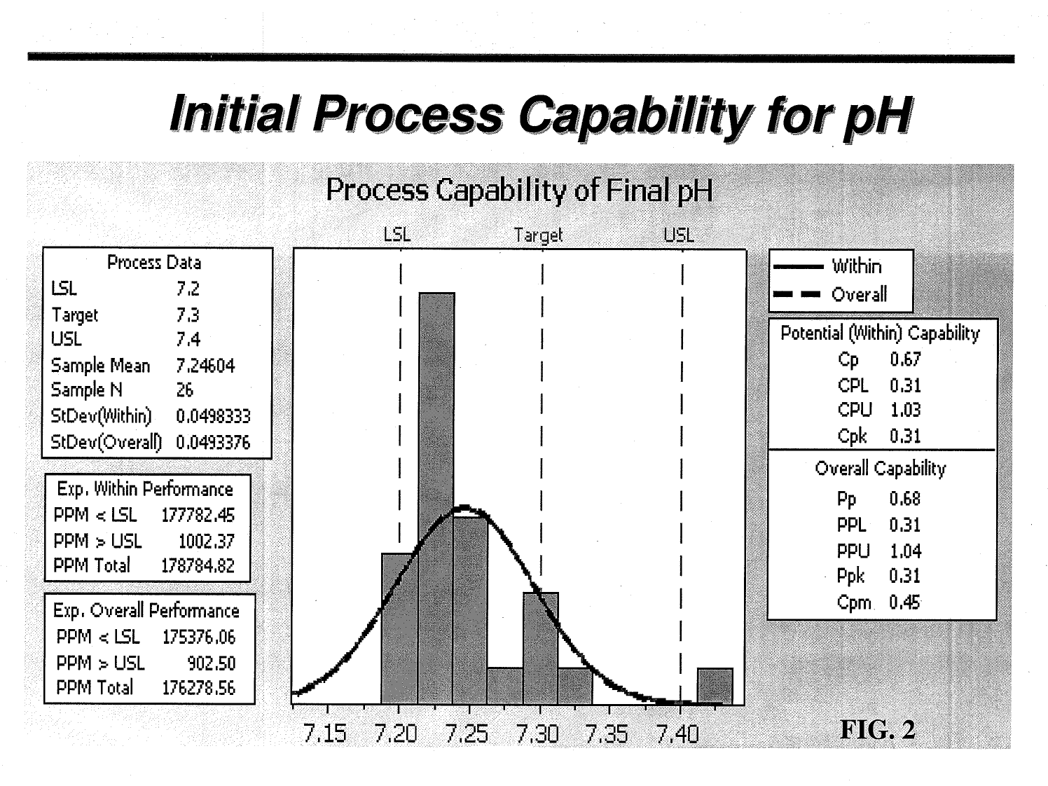

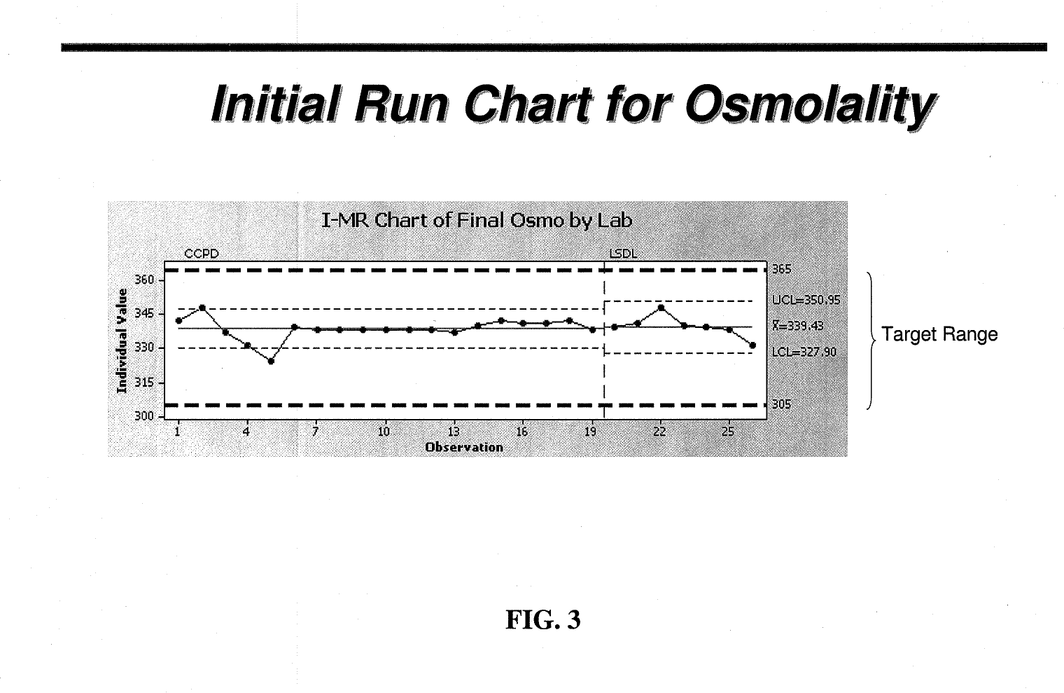

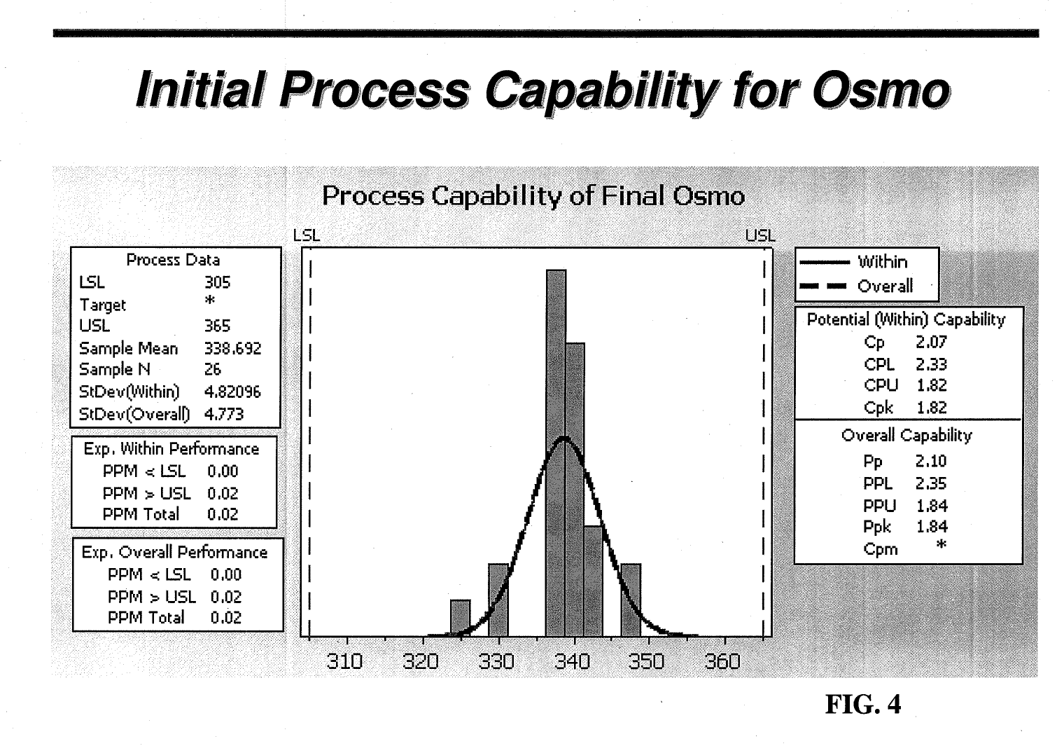

FIGS. 1-4 depict initial run chart for pH, initial process capability for pH, initial run chart for osmolality, and initial process capability for osmolality, respectively.

“Define” Step

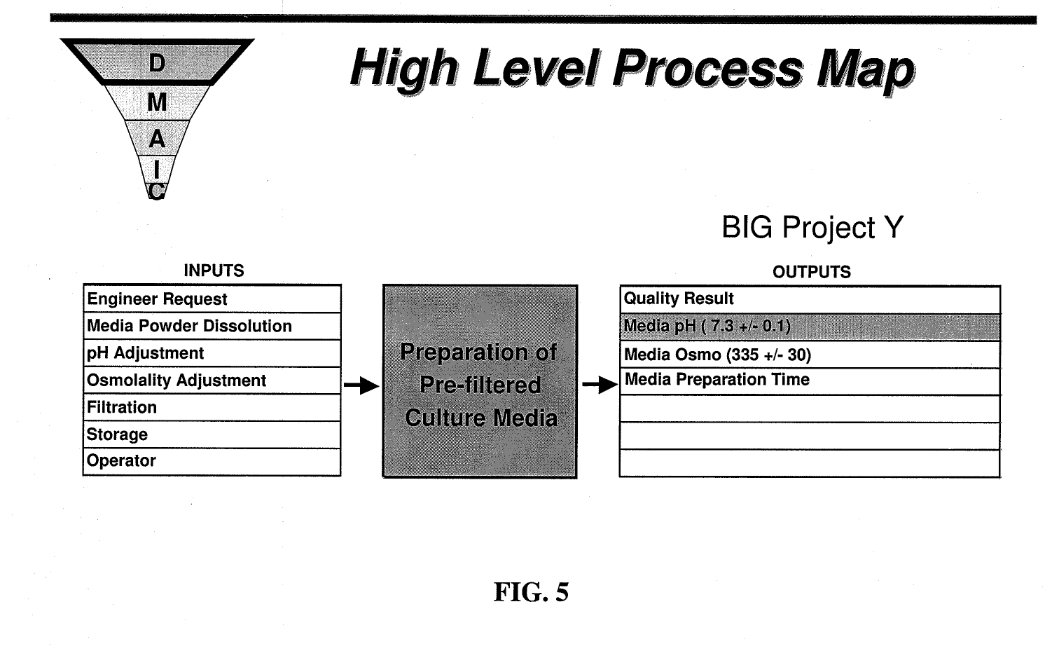

FIG. 5 depicts a high level process map. Other items that can be used as inputs include raw materials, measurement, method, equipment, and variation. The culture media preparation process can include the steps of measurement, dissolution, adjustment, and filtration. Other factors such as controllable variables and uncontrollable variables can also affect the process.

The goal of the defining step is to ensure the media preparation step will result in media that meet the specifications and to reduce the amount of time for media preparation by minimizing the secondary adjustment.

### “Measure” Step

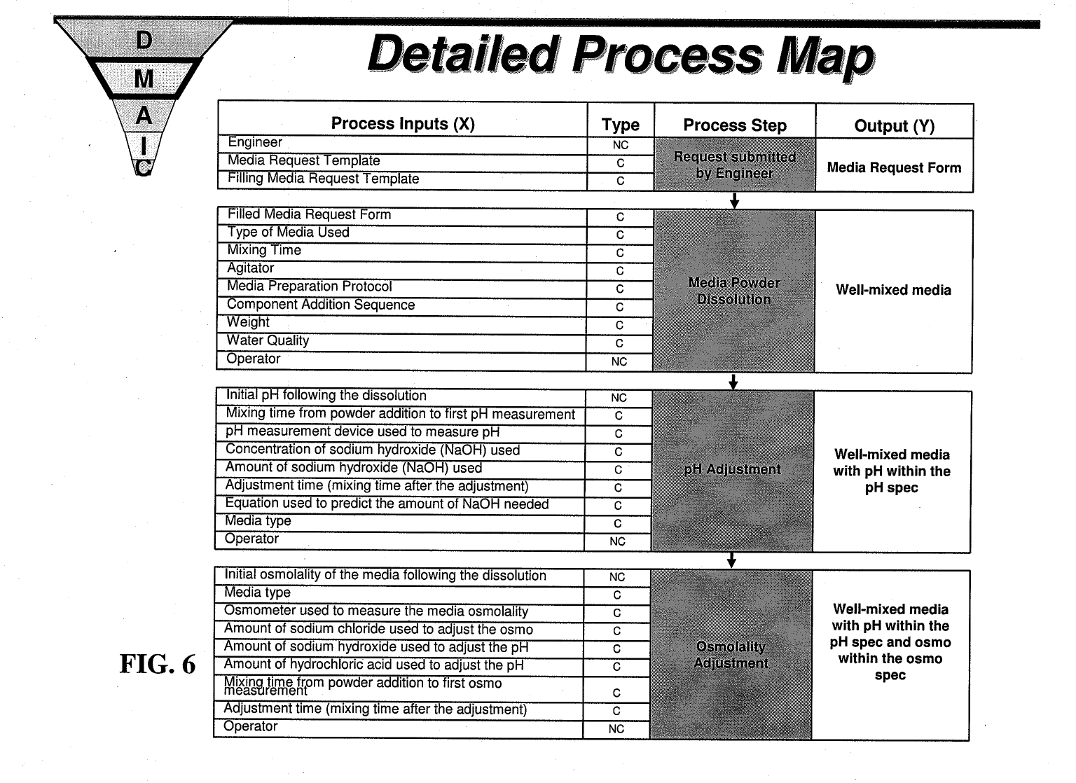

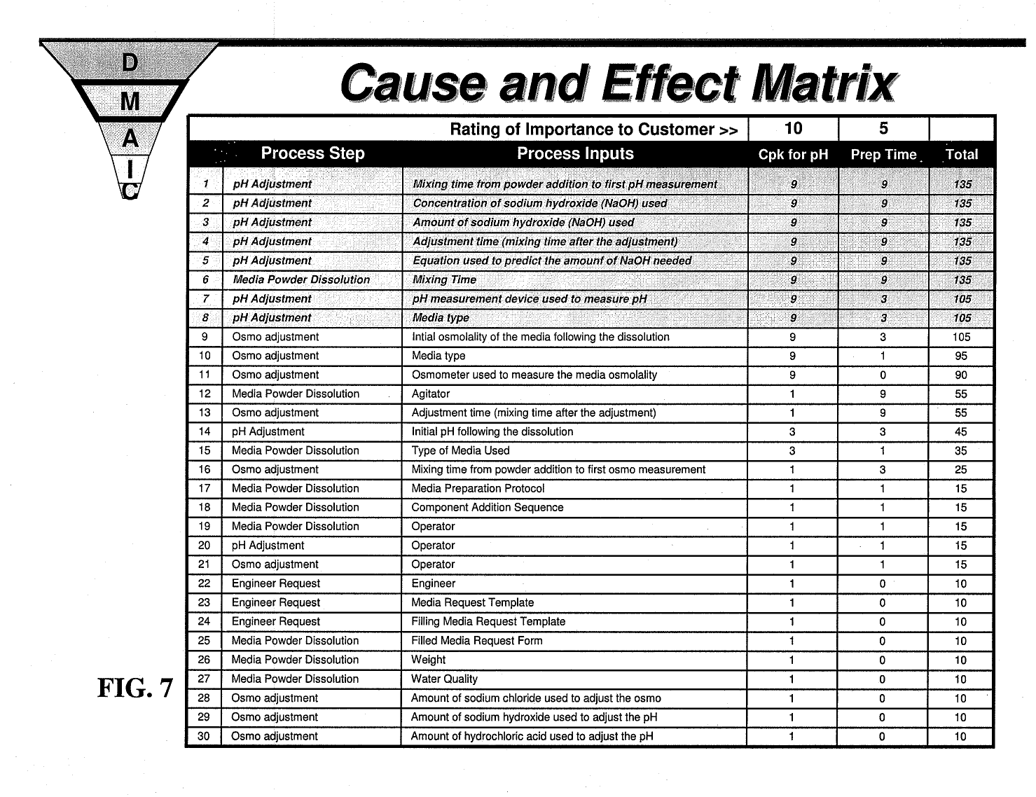

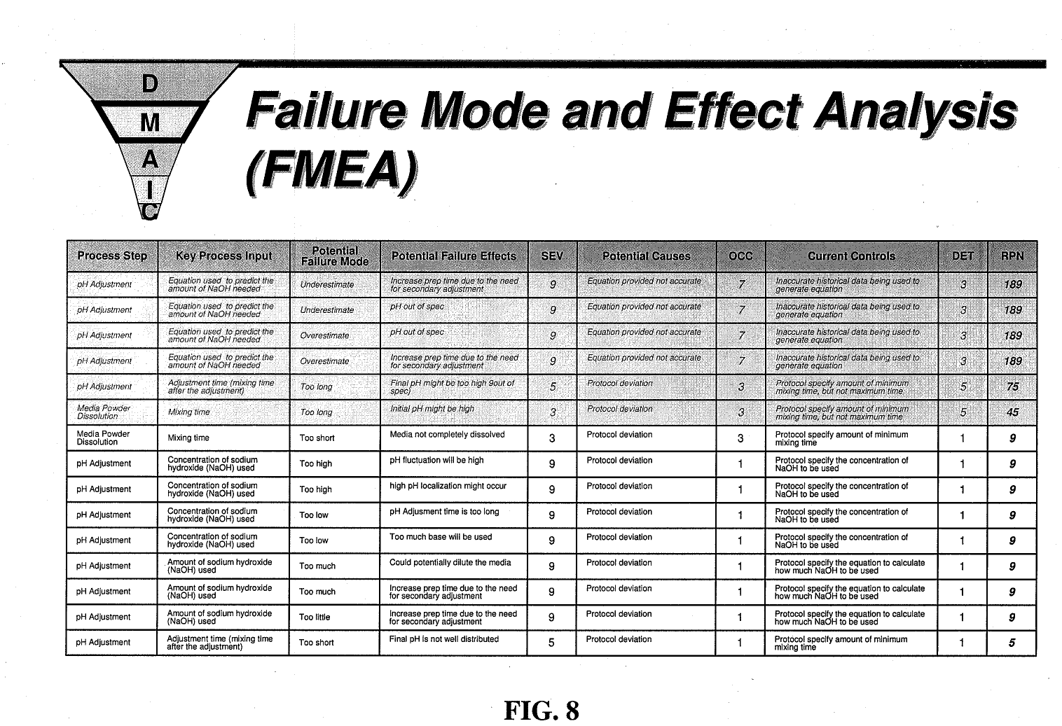

FIGS. 6-8 depict detailed process map, cause and effect matrix, and failure mode and effect analysis (FMEA), respectively.

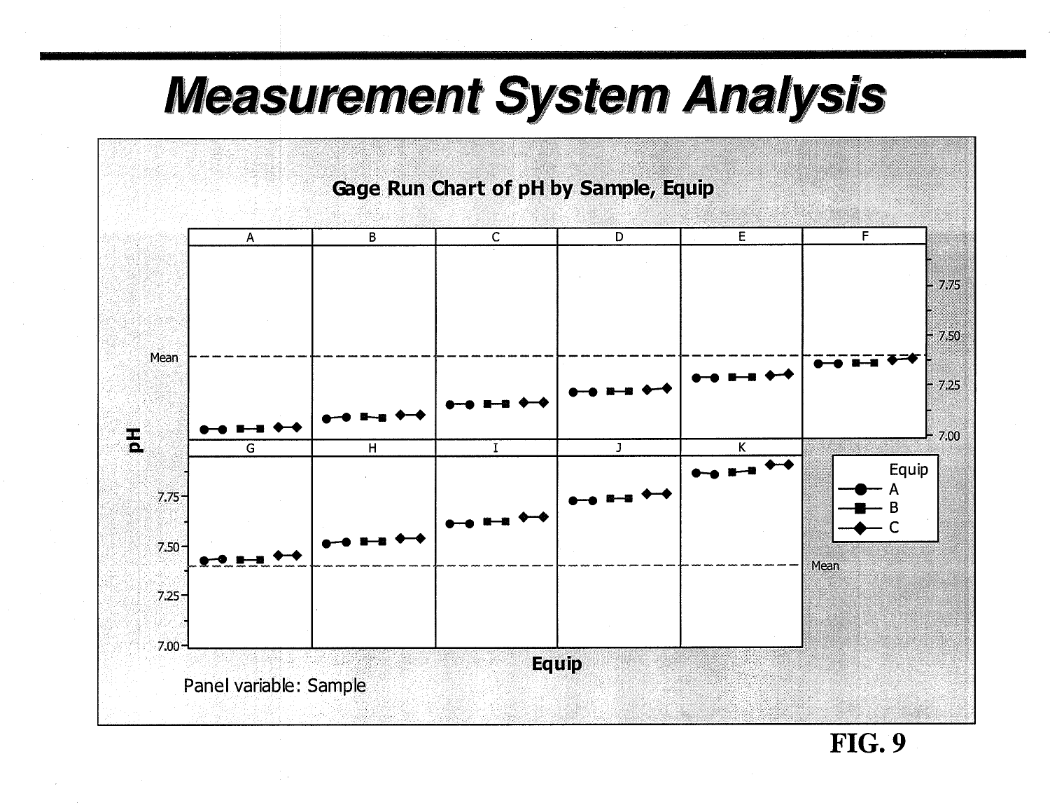

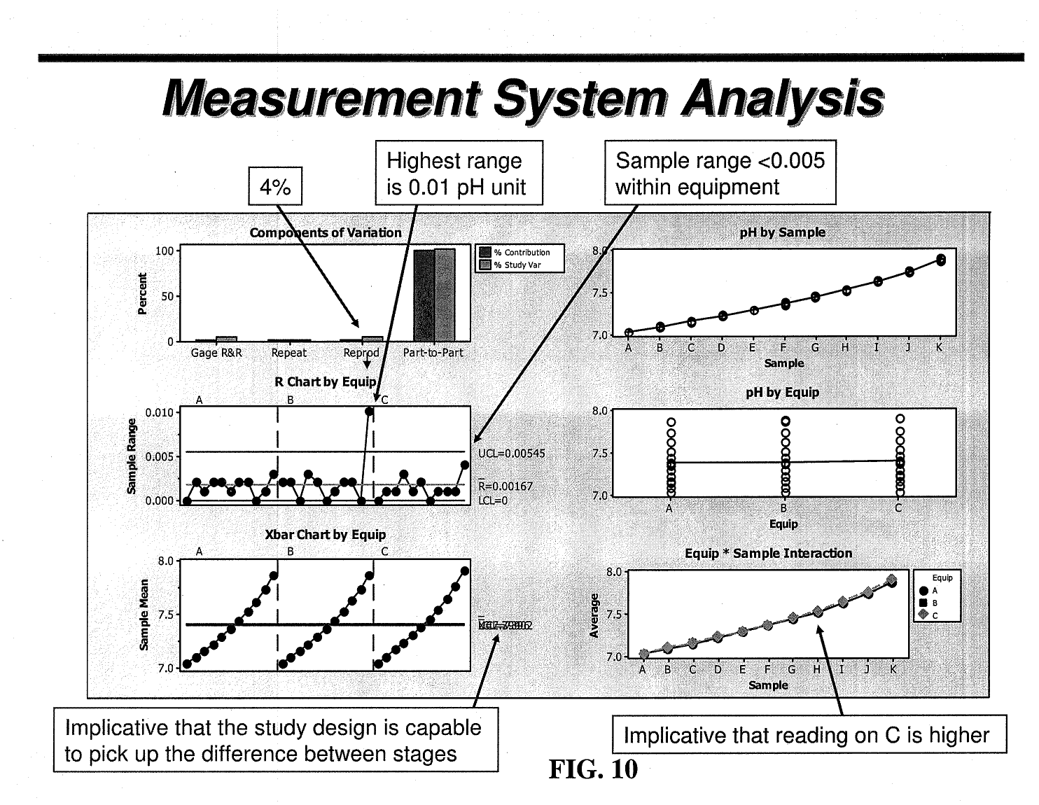

The current media preparation protocol did not specify which measurement instrument to use. The assumption is that all equipment should give comparable results. A measurement system analysis (MSA) was performed by preparing samples with 11 (eleven) different pHs and randomly subjected to pH readings on three different pH measuring devices. The results for the measurement system analysis are shown in FIGS. 9-10.

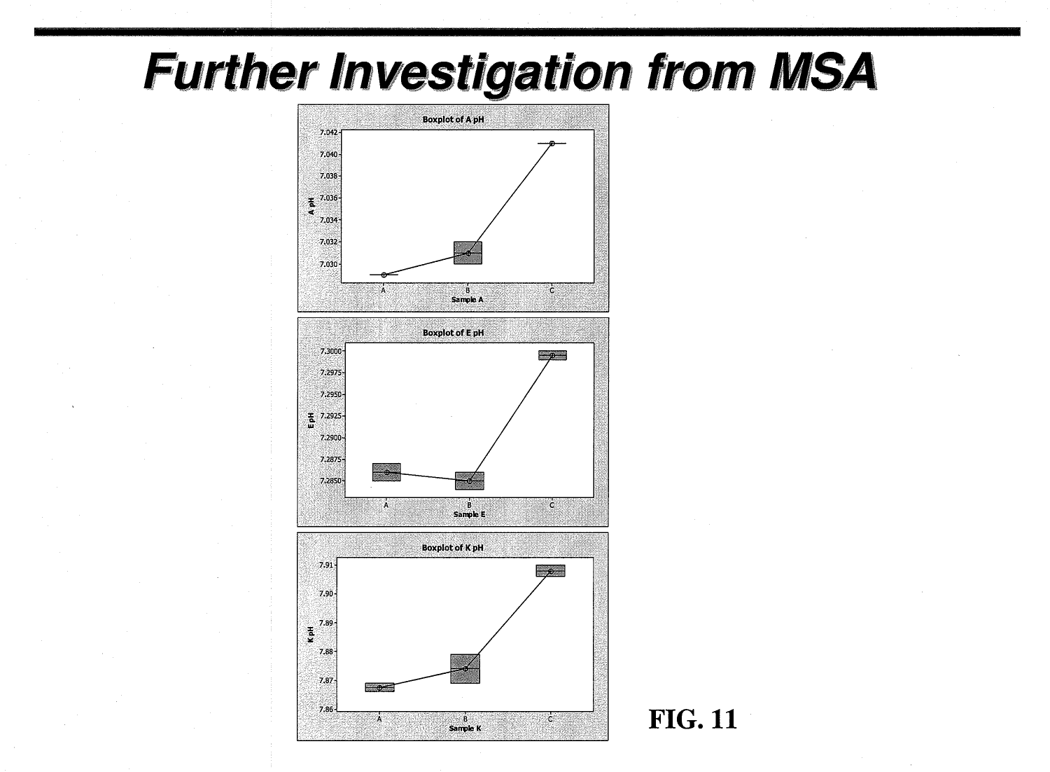

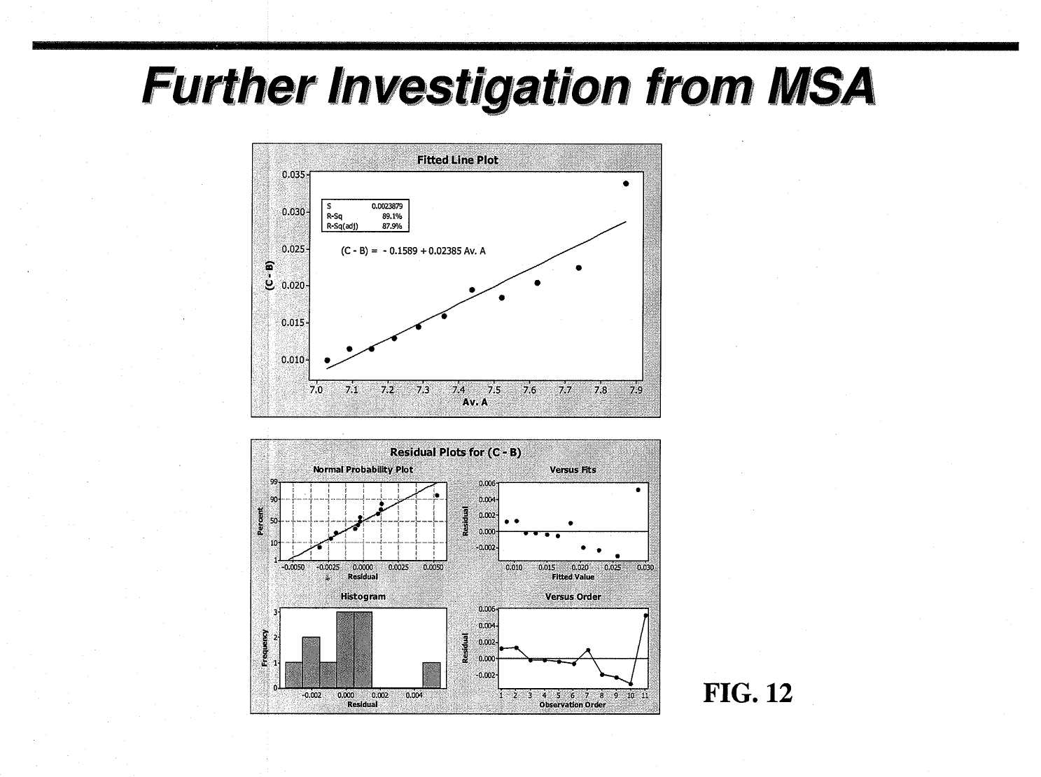

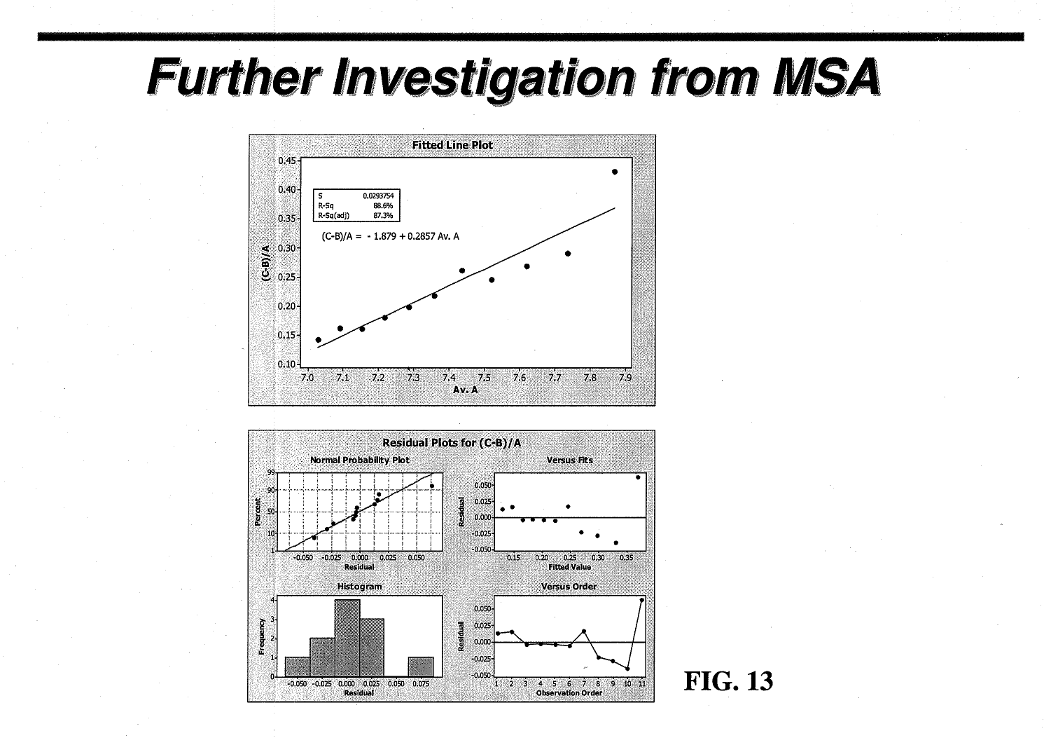

Analysis of variance (ANOVA) was performed to compare the pH between samples from three different reading instruments. Representative box-plots from the low pH, mid pH, and high pH samples are shown in FIG. 11. As shown in FIG. 11, Instrument C was reading a higher pH than Instrument A or B for sample pH between 7.00 to 7.80. This might be the source of variation during the MSA, although 4% variation for MSA is acceptable. As shown in FIG. 12, there was a strong correlation between sample pH and the pH difference (p-value <0.001; R2>89%) when pH reading from Instrument A was used as the base reading for sample pH. This result implicates that the higher the sample pH, the higher the variation. Further, as shown in FIG. 13, although the % Diff is low (>0.45%), the % Diff was shown to be a function of sample pH when pH reading from Instrument A was used as the base reading for sample pH. This results implicates that the higher the sample pH, the higher the relative/percent variation.

### “Analyze” Step

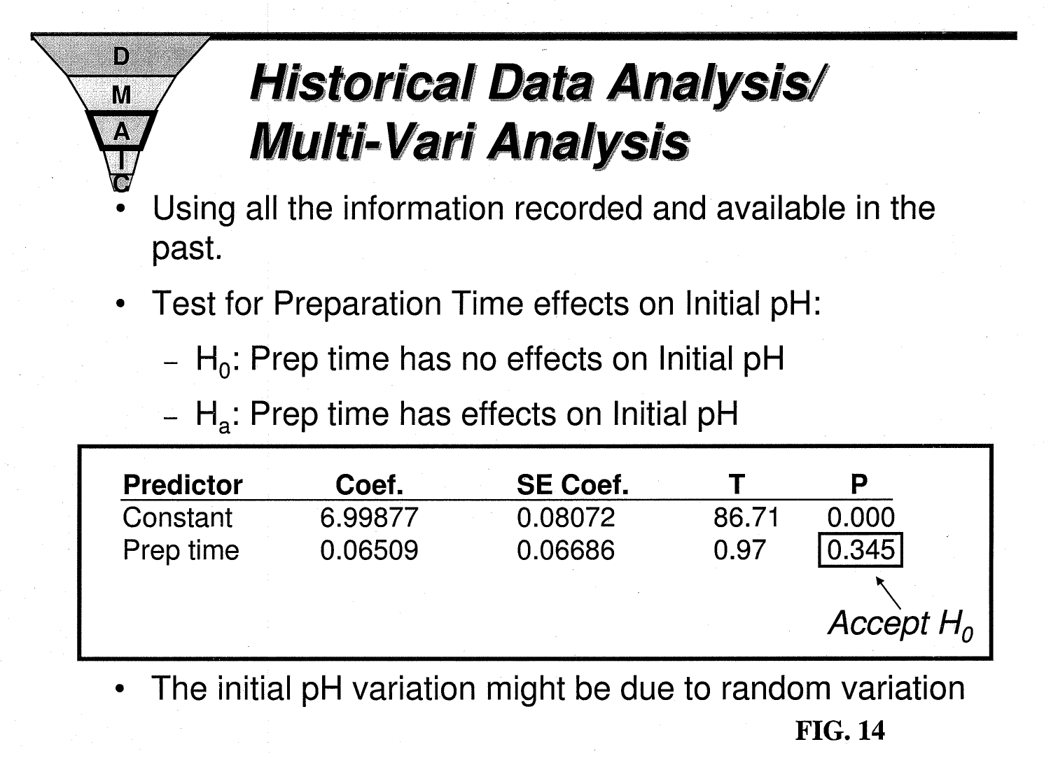

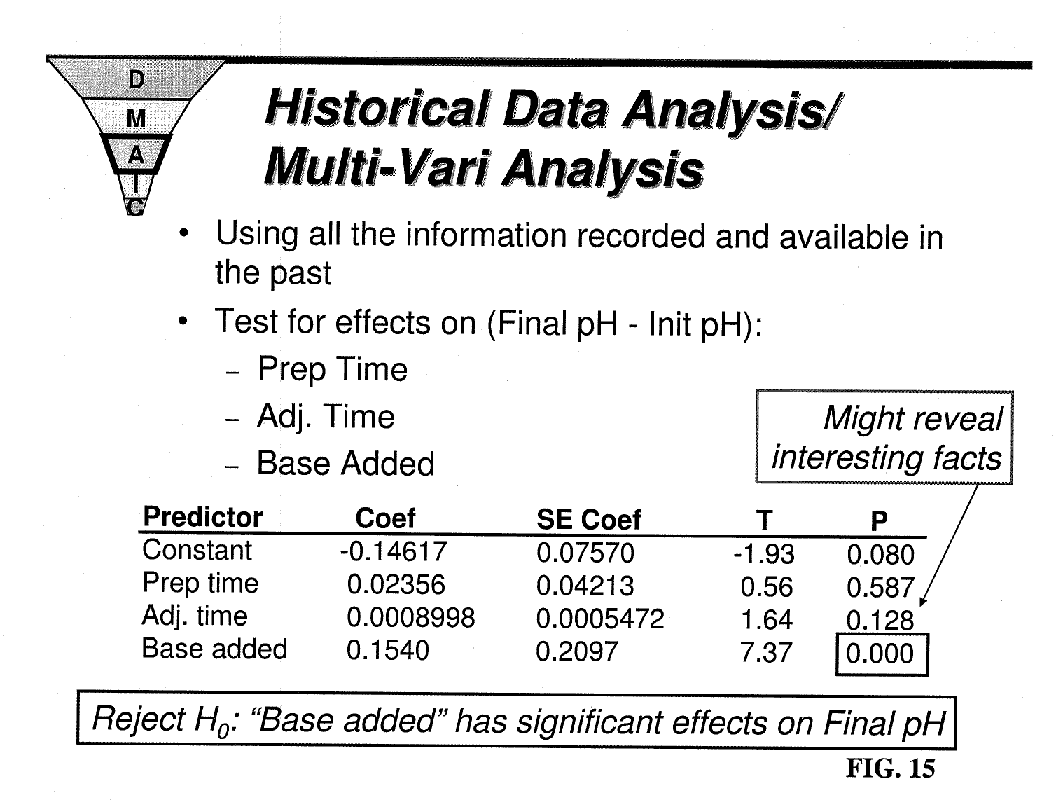

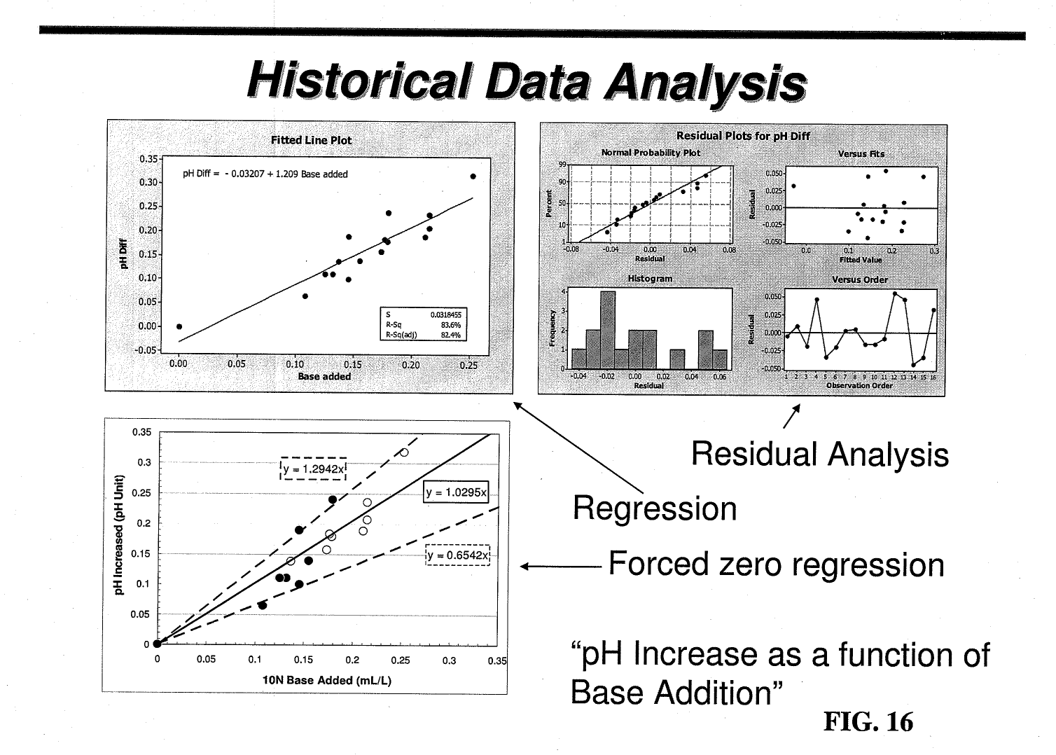

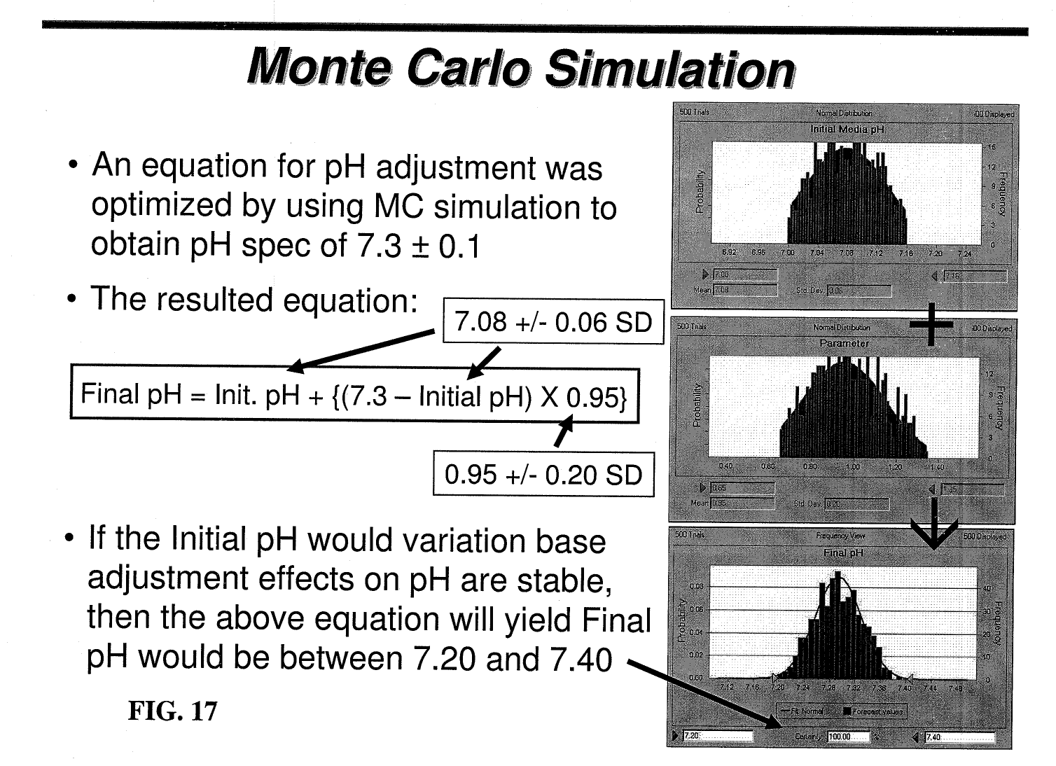

The results for historical data analysis and multivariate analysis are shown in FIGS. 14-16. The results for Monte Carlo simulation are shown in FIG. 17.

### “Improve” Step

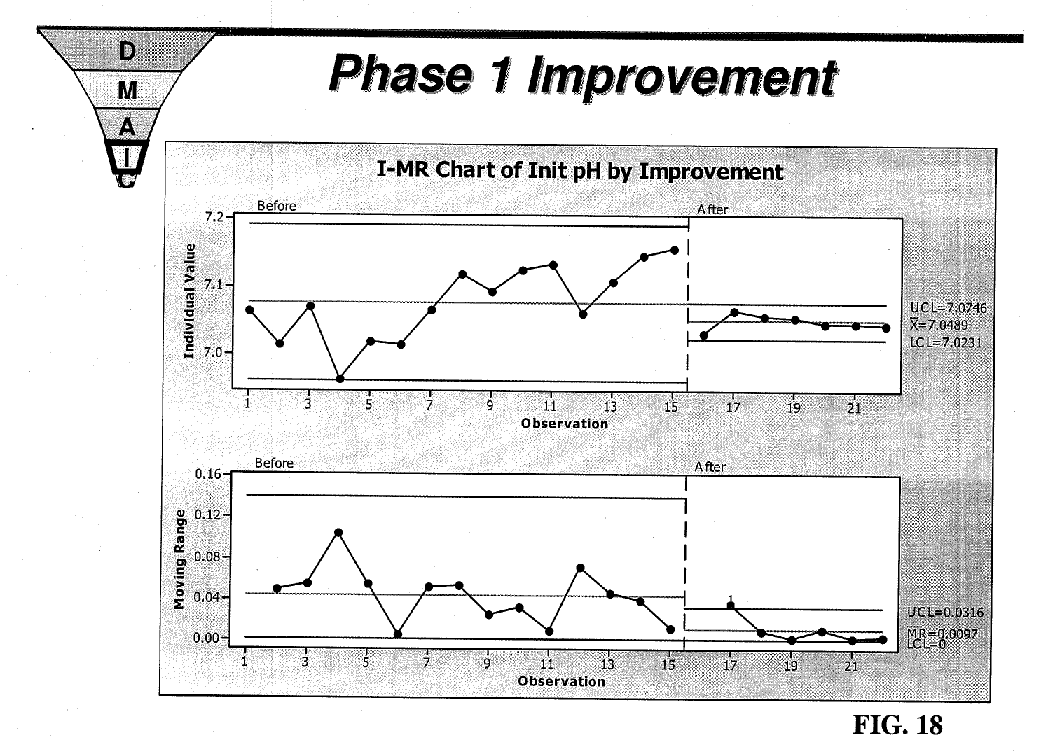

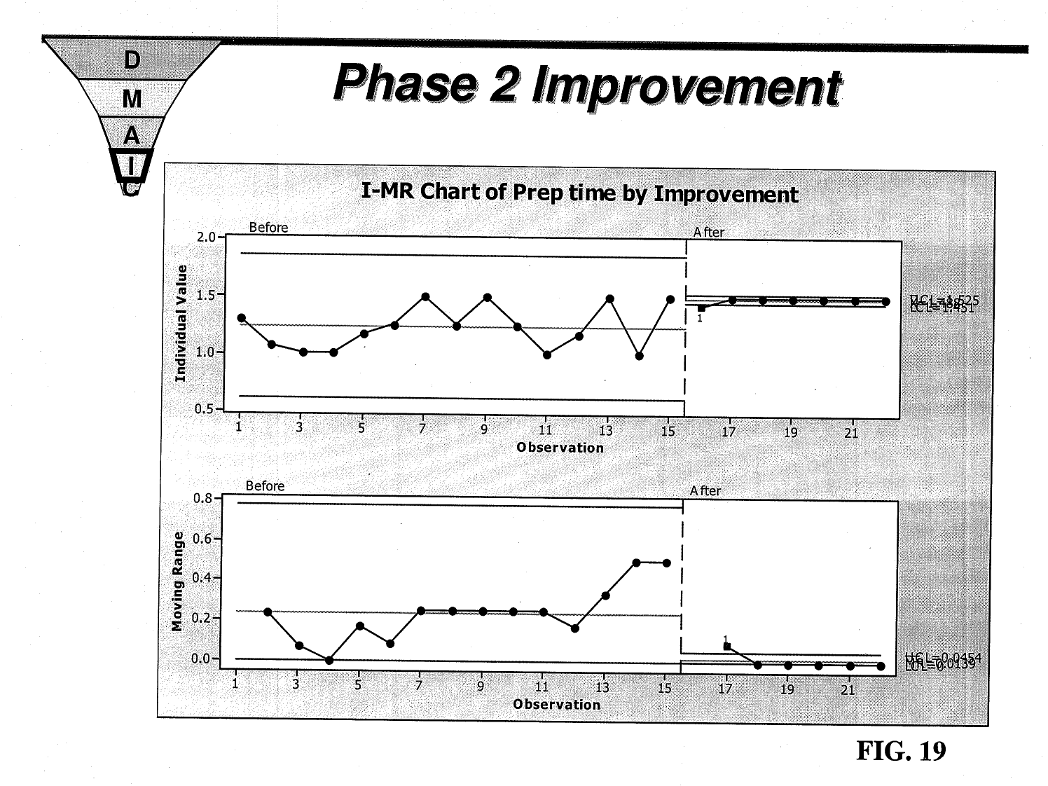

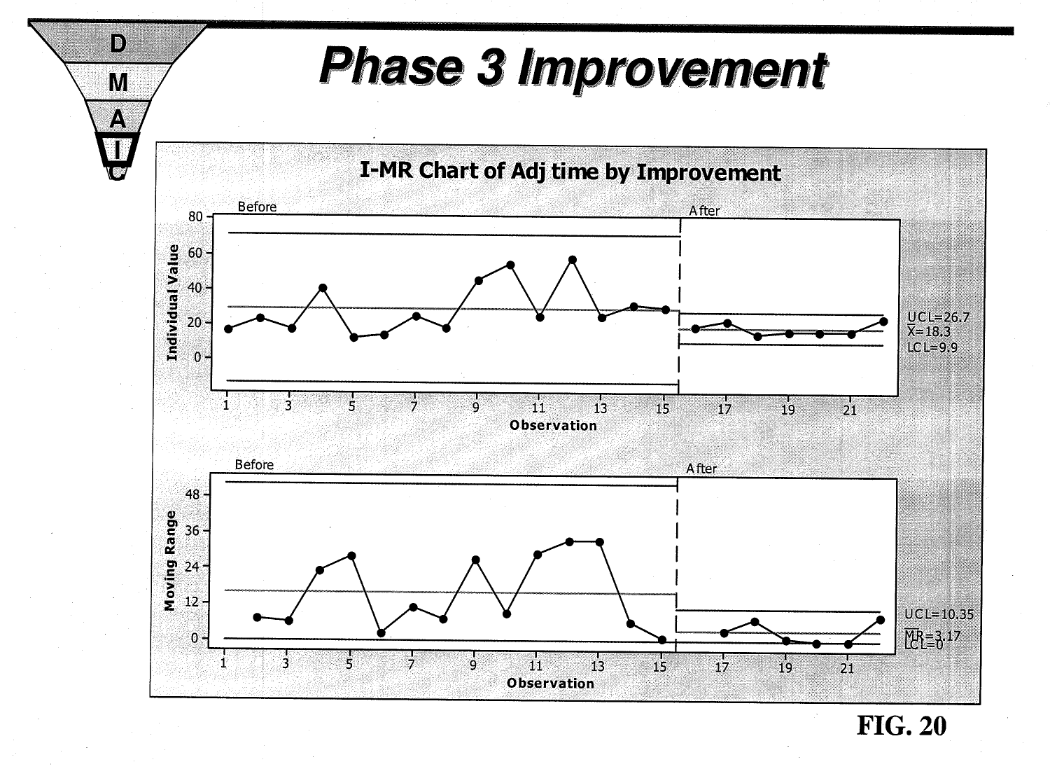

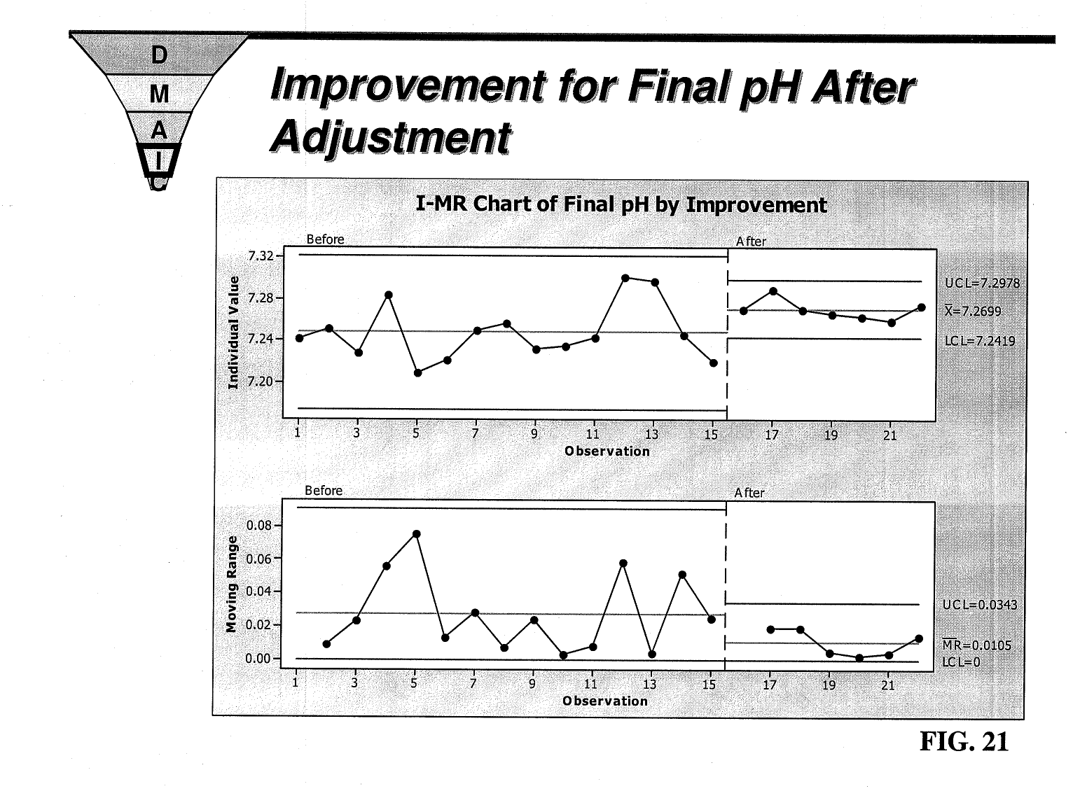

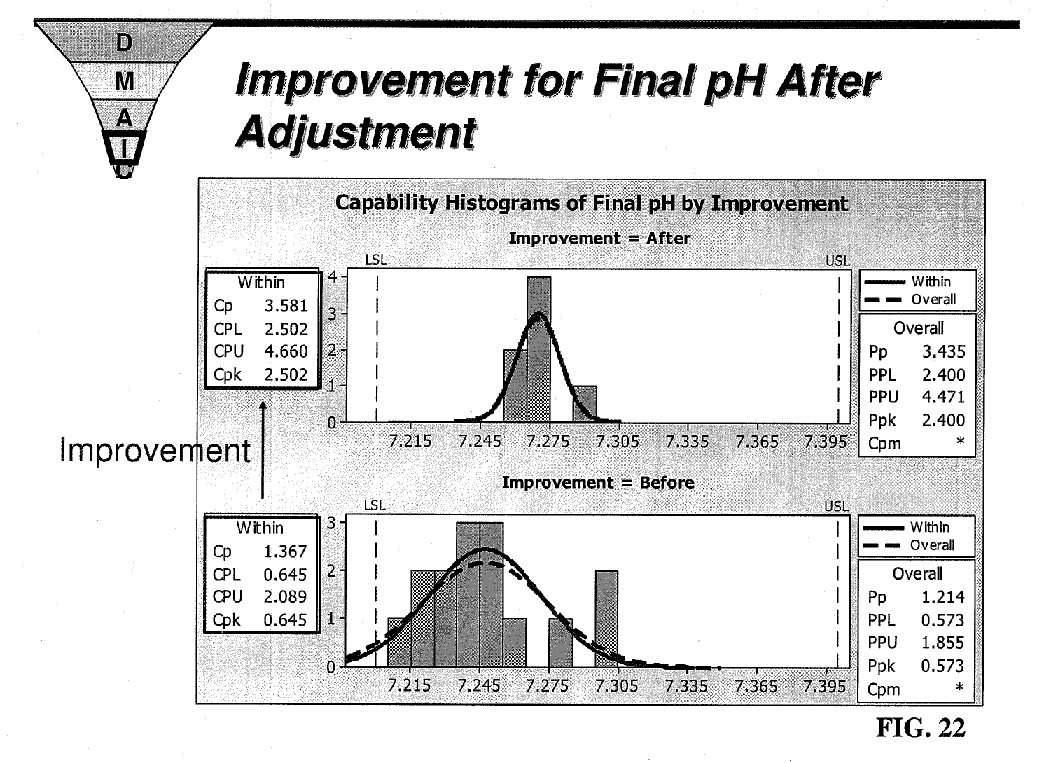

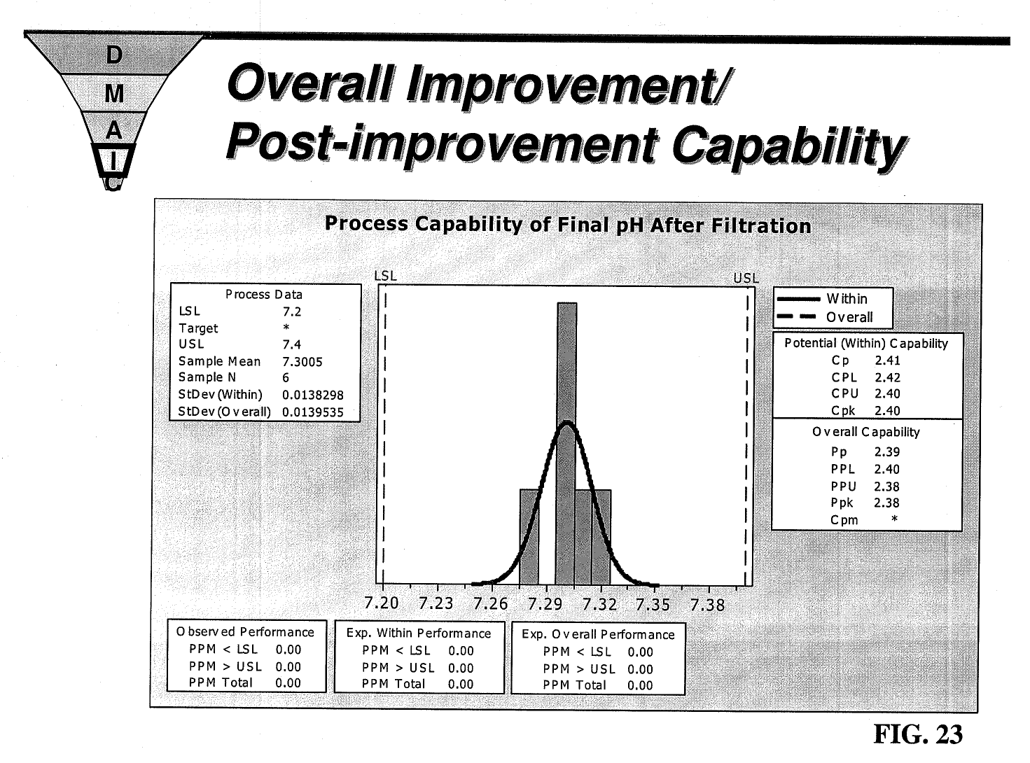

The improvement plan includes four phases. Phase 1 is to control the pH measurement by using only one pH measurement system for both initial pH reading and final pH reading. Phase 2 is to control the Total Mixing Time (between dissolution and the first pH measurement) to be between 1 hour and 1.5 hour. Phase 3 is to control the Adjustment Time (between first sample pH measurement and after the pH adjustment) to be between 15 and 30 minutes. Phase 4 is to improve the pH adjustment guideline equation to: Amount of 10 N NaOH added=(7.3−Initial pH)×0.90. FIGS. 18-20 shows Phase 1 to Phase 3 improvements, respectively. FIGS. 21-22 show improvement for final pH after adjustment. FIG. 23 shows overall improvement and post-improvement capability.

### “Control” Step

The control plan includes correction for the pH adjustment equation in the media preparation protocol; controlling and recording the media preparation mixing time to be between 1 and 1.5 hours; controlling and recording the mixing time after the media adjustment to be between 15 and 30 minutes; and monitoring the media osmolality after the pH adjustment.

The control plan can also include monitoring the capability of the pH adjustment equation to adjust pH within the specifications; monitoring the variation of the initial pH after the improvement; and monitoring the variation of pH adjustment equation after controlling the pH adjustment time.

In conclusion, the process capability (Cpk) for the culture media preparation was improved from 0.65 to 2.5 through improvement and control plan. Six Sigma approach can be suitably applied for process improvement trouble shooting in cell culture media preparation steps. Monte Carlo simulation is a powerful tools to quantify variation and could be used to estimate variation for process improvement.

1. A method of producing a recombinant protein, the method comprising:
preparing a culture medium comprising mixing medium powder in water for about 1-1.5 hours,
adjusting medium pH to about 7.2-7.4, and
adjusting medium osmolality to about 305-365 Osm/L; and
culturing a cell under conditions that allow the expression of the recombinant protein, to thereby producing the recombinant protein.

2. The method of claim 1, wherein the protein is arylsulfatase A.

3. The method of claim 1, wherein the cell is a mammalian cell.

4. The method of claim 1, wherein preparing a culture medium further comprises mixing the culture medium after adjusting medium pH for about 15-30 minutes.

5. The method of claim 1, wherein preparing a culture medium further comprises mixing the culture medium after adjusting medium osmolality for about 15-30 minutes.

6. The method of claim 1, wherein adjusting medium pH is performed prior to adjusting medium osmolality.

7. The method of claim 1, further comprising improving the process capability of the culture medium preparation.

8. The method of claim 7, wherein the process capability is determined by a process capability index or a process performance index.

9. The method of claim 8, wherein the process capability index is improved from about 0.65 to about 2.50.

10. A method of preparing a culture medium, the method comprising:
mixing medium powder in water for about 1-1.5 hours;
adjusting medium pH to about 7.2-7.4; and
adjusting medium osmolality to about 305-365 Osm/L,
to thereby preparing the culture medium.

11-16. (canceled)

17. The method of claim 7, wherein improving the process capability of the culture medium preparation comprises defining one or more characteristics of the culture medium preparation, and wherein the characteristics comprise medium pH, medium osmolality, medium preparation time, or a combination thereof.

18. (canceled)

19. The method of claim 7, wherein improving the process capability further comprises measuring one or more parameters associated with the culture medium preparation, and wherein the parameter is associated with medium powder dissolution, pH adjustment, or osmolality adjustment.

20. (canceled)

21. The method of claim 19, wherein the parameter associated with medium powder dissolution is selected from the group consisting of type of medium, mixing time, type of agitator, type of medium preparation protocol, component addition sequence, weight, and water quality.

22. The method of claim 19, wherein the parameter associated with pH adjustment is selected from the group consisting of: initial pH following the dissolution, mixing time from powder addition to first pH measurement, pH measurement device used to measure pH, concentration of sodium hydroxide (NaOH) used, amount of sodium hydroxide (NaOH) used; mixing time after the pH adjustment, equation used to predict the amount of sodium hydroxide needed, and type of medium.

23. The method of claim 19, wherein the parameter associated with osmolality adjustment is selected from the group consisting of: initial osmolality of the medium following the dissolution, type of medium, osmometer used to measure the medium osmolality, amount of sodium chloride used to adjust the osmolality, amount of sodium hydroxide used to adjust the pH, amount of hydrochloric acid used to adjust the pH, mixing time from powder addition to first osmolality measurement, and mixing time after the adjustment.

24. The method of claim 19, wherein measuring one or more parameters comprises
performing a failure mode and effect analysis, or
performing a measurement system analysis.

25. (canceled)

26. The method of claim 7, wherein improving the process capability further comprises analyzing one or more parameters associated with the culture medium preparation, and wherein analyzing one or more parameters comprises performing a historical data analysis, a multivariate analysis, a Monte Carlo simulation, or a combination thereof.

27. (canceled)

28. The method of claim 7, wherein improving the process capability further comprises improving one or more parameters associated with the culture medium preparation, and wherein improving one or more parameters comprises improving the pH measurement, improving the total mixing time, improving the adjustment time, improving the pH adjustment guideline equation, or a combination thereof.

29. (canceled)

30. The method of claim 7, wherein improving the process capability further comprises controlling one or more parameters associated with the culture medium preparation.

31. The method of claim 30, wherein controlling one or more parameters comprises correcting for the pH adjustment equation in the medium preparation protocol, controlling or recording the medium preparation mixing time, controlling or recording the mixing time after the medium adjustment, monitoring the medium osmolality after the pH adjustment, or a combination thereof.

32. The method of claim 31, wherein controlling one or more parameters further comprises monitoring the capability of the pH adjustment equation to adjust pH within specs, monitoring the variation of the initial pH after the improvement, monitoring the variation of pH adjustment equation after controlling the pH adjustment time, or a combination thereof.

33. A method of culturing a cell, the method comprising:
preparing a culture medium comprising mixing medium powder in water for about 1-1.5 hours,
adjusting medium pH to about 7.2-7.4, and
adjusting medium osmolality to about 305-365 Osm/L; and
contacting a cell with the culture medium under conditions that allow culturing of the cell, to thereby culturing the cell.

34-40. (canceled)

41. A protein produced by the method of claim 1.

42. A culture medium prepare by the method of claim 10.
